(19) United States
(12) Patent Application Publication
(10) Pub. No.: US 2010/0022600 A1
(43) Pub. Date: Jan. 28, 2010

(54) METHODS AND COMPOSITIONS FOR TREATING CENTRAL NERVOUS SYSTEM TUMORS

(76) Inventors: Gary E. Landreth, Shaker Heights, OH (US); Christian Grommes, Willoughby Hills, OH (US)

Correspondence Address: TAROLLI, SUNDHEIM, COVELL & TUMMINO, LLP, 1300 EAST NINTH STREET, SUITE 1700, CLEVELAND, OH, 44114, US

(21) Appl. No.: 12/439,395

(22) PCT Filed: Aug. 28, 2007
(86) PCT No.: PCT/US07/76995

§ 371 Date: Feb. 27, 2009

Related U.S. Application Data
(60) Provisional application No. 60/840,673, filed on Aug. 28, 2006.

(51) Int. Cl. A61K 31/4439; A61K 31/41; A61K 31/427; A61K 31/352; A61P 35/00

(52) U.S. Cl. 514/342; 514/360; 514/369; 514/456

(57) ABSTRACT

A method of treating a central nervous system tumor in a subject includes administering a therapeutically effective amount of at least one PPARγ agonist or a derivative thereof to the tumor in the subject.

### RELATED APPLICATION

This application claims priority from U.S. Provisional Application No. 60/840,673, filed Aug. 28, 2006, the subject matter, which is incorporated herein by reference.

### FIELD OF THE INVENTION

The present invention relates to methods and compositions used for treating central nervous system tumors and particularly relates to methods and compositions for treating brain tumors.

### BACKGROUND

Malignant astrocytic gliomas are the most common primary brain tumors. Glioma cells show a high proliferation rate and diffusely infiltrate adjacent brain tissue (J Neuropathol Exp Neurol 2002; 61:215-25). These tumors initially respond to radiation and to a lesser degree to chemotherapy, however, they invariably recur. Despite substantial efforts, no curative therapy exists and median overall-survival of the most malignant variant “glioblastoma” is still within one year (N Engl J Med 2001; 344: 114-23).

Peroxisome proliferator-activated receptor γ (PPARγ) are a subclass of nuclear hormone receptors, enabling the cell to respond to extracellular stimuli by transcriptionally regulating gene expression. Three isoforms of PPARs have been identified and designated as -α, -βδ, and -γ. All are encoded by different genes. PPARs form heterodimers with the retinoic acid receptor (RXR) and exhibit ligand-induced transcriptional regulatory activity through sequence-specific PPAR-responsive elements (PPRE) in their target genes (J Med Chem 2000; 43: 527-50). For more than a decade work on PPARs was driven by their important role in the regulation of cellular metabolism, especially in tissues known for high rates of β-oxidation such as liver, heart, muscle and kidney. Since activation of the PPARγ subtype results in reduced serum glucose (Annu Rev Cell Dev Biol 1996; 12: 335-63) recently developed synthetic PPARγ agonists are already in clinical use as anti-diabetic drugs (pioglitazone (ACTOS); rosiglitazone (AVANDIA)).

Apart from well defined metabolic actions, PPARγ agonists exhibit several antineoplastic effects (Lancet Oncol 2004; 5: 419-29) and induce apoptotic cell death in various malignant cell lineages, including liposarcoma (Proc Natl Acad Sci USA 1997; 94: 237-41), breast adenocarcinoma (Proc Natl Acad Sci USA 1998; 95:8806-11; Mol Cell 1998; 1: 465-70), prostate carcinoma (Cancer Res 1998; 58: 3344-52), colorectal carcinoma (Gastroenterology 1998; 115: 1049-55; Nat Med 1998; 4: 1046-52), non-small cell lung carcinoma (Cancer Res 2000; 60: 1129-38), pancreatic carcinoma (Cancer Res 2000; 60: 5558-64), bladder cancer (Neoplasia 1999; 1: 330-9), and gastric carcinoma (Br j Cancer 2000; 83: 1394-400). A recent study has shown PPARγ agonist mediated reduction of glioma cell survival caused by an increased production of reactive oxygen species (J Biol Chem 2004; 279: 8976-85). Furthermore, PPARγ agonists moderately inhibited growth of BT4Cn rat glioma cells, an effect which was abolished by the PPARγ antagonist GW9662 (Carcinogenesis 2001; 22: 1747-55). A significant proportion, 95% of glioma tissues from 20 patients expressed PPARγ mRNA (Jpn J Cancer Res 2002; 93: 660-6).

### SUMMARY OF THE INVENTION

The present invention relates to a method of treating central nervous system tumors in a subject. The central nervous system tumors, can include, for example, brain tumors, gliomas, glioblastoma multiforme, neuroblastomas, medullablastomas as well as other very aggressive central nervous system tumors. In the method, a therapeutically effective amount of at least one PPARγ agonist or a derivative thereof is administered to a central nervous system tumor of the subject. The PPARγ agonist or derivative thereof can be administered to the tumor cells (e.g., glioma cells) in a subject's central nervous system at an amount effective to induce upregulation of proapoptotic protein Bax and cleaved caspase-3, which is indicative of apoptotic cell death. In another aspect, the PPARγ agonists or derivative thereof can be administered to the tumor in an amount effective to reduce or suppress expression of MMP-9, a protein that is associated with glioma invasion. Furthermore, PPARγ agonists or derivative thereof can be administered to tumor in an amount effective to induce upregulation of the astrocytic redifferentiation marker CS-56 in the tumor cells.

The PPARγ agonist or derivative thereof can be administered orally and/or by intracerebral infusion to the subject. The intracerebral infused PPARγ agonist or derivative thereof can be provided in dimethyl sulfoxide solution. The amount of PPARγ agonist or derivative thereof administered to the subject can depend on the specific PPARγ agonist or derivative thereof selected. For example, pioglitazone can be administered to a subject at a dose of about 40 mg/kg per day.

In one aspect of the invention the PPARγ agonist or a derivative thereof comprises thiazolidinedione or a derivative thereof. In another aspect of the invention, the PPARγ agonist or a derivative thereof comprises at least one compound or a pharmaceutically salt thereof selected from the group consisting of (+)-5[[4-[(3,4-dihydro-6-hydroxy-2,5,7,8-tetramethyl-2H-1-benzopyran-2-yl)methoxy]phenyl]methyl]-2,4thiazolidinedione; 5-[4-[2-(5-ethylpyridin-2-yl)ethoxyl]benzyl]thiazolidine-2,4-dione; 5-[4-[(1-methylcyclohexyl)methoxy]benzyl]thiazolidine-2,4-dione; (ciglitazone); 4-(2-naphthylmethyl)-1,2,3,5-oxathiadiazole-2-oxide; 5-[4-[2-[(N-(benzoxazol-2-yl)-N-methylamino]ethoxy]benzyl]-5-methlthiazolidine-2,4-dione; 5-[4-[2-[2,4dioxo-5-phenylthiazolidine-3-yl)ethoxy]benzyl]thiazolidine-2,4-dione; 5-[4-[2-[(N-methyl-N-(phenoxycarbonyl)amino]ethoxy]benzyl]thiazolidine-2,4-dione; 5-[4-[2-phenoxyethoxy)benzyl]thiazolidine-2,4-dione; 5-[4-[2-(4-chorophenyl)ethylsulfonyl]benzyl]thiazolidine-2,4-dione; 5-[4-[3-(5-methyl-2-phenyloxazol-4-yl)propionyl]benzyl]thiazolidine-2,4-dione; 5-[[4-(3-hydroxy-1-methylcyclohexyl)methoxy]benzyl]thiazolidine-2,4-dione; 5-[4-[2-(5-methyl-2-phenyloxazol-4-yl)ethoxyl]benzyl]thiazolidine-2,4-dione; 5-[(2-benzyl-2,3-dihydrobenzopyran)-5-ylmethyl]thiazolidine-2,4-dione; 5-[[2-(2-naphthylmethyl)benzoxazol]-5-ylmethyl]thiazolidine-2,4-dione; 5-[4-[2-(3-phenylureido)ethoxyl]benzyl]thiazolidine-2,4-dione; 5-[4-[2-(N-benzoxazol-2-yl)-N-metholamino]ethoxy]benzyl]thiazolidine-2,4-dione; 5-[4-[3-(5-methyl-2-phenyloxazol-4-yl)propionyl]benzyl]thiazolidine-2,4-dione; 5-[2-(5-methyl-2-phenyloxazol-4-ylmethyl)benzofuran-5-ylmethyl]oxazolidine-2,4-dione; 5-[4-[2-(N-methyl-N-(2-pyridyl)amino]ethoxy]benzyl]thiazolidine-2,4-dione; and 5-[4-[2-(N-(benzoxazol-2-yl)-N-methylamino]ethoxy]benzyl]oxazolidine-2,4-dione.

### BRIEF DESCRIPTION OF THE DRAWINGS

FIG. 1 illustrates Pioglitazone reduced cellular viability of rat and human glioma cells and proliferation in vitro. Cellular viability of human glioma cells A172 after transfection with vector control, two different PPARγ-cDNA's and PPARγ mutant cDNA (E499Q) and 5 d of pio treatment (A). Viability of rat C6 glioma cells (B), human U87 and A172 glioma cells (C) or primary astrocytes (D) at 1, 3, 5, 7 d incubated with the PPARγ agonist pioglitazone (pio) (1, 10, 30 μM for C6 and primary astrocytes; 30 μM for U87 and A172) using the MU-assay. Data are expressed as percentage viable cells relative to untreated control cultures. Asterisks indicate significant level in student-T-test: ***p<0.0001 (n=10).

FIG. 2 illustrates intracerebral and oral administration of pioglitazone significantly reduced tumor growth in the rat glioma model. C6 glioma cells were injected in rat striata and treated with pio (20 μM) or vehicle (DMSO) via an osmotic pump over 3 d (n=5/group), 6 d (n=5/group), 9 d (n=5/group), 14 d (n5/group), and 21 d (n=6/group). Pio-treated animals showed significant smaller tumor volumes (B, D, E, F) compared to untreated animals (A, C, E, F). A, C: Striatum of a vehicle-treated animal at 21 d, HE-staining, A: Bar: 1 mm; C: Bar: 100 μm. B, D: Striatum of a pio-treated animal at 21 d, HE-staining, B: Bar: 1 mm; D: Bar: 100 μm. E: Representation of tumor area in treated (pio) and untreated (vehicle) animals at 21 d. Black: tumor tissue. Graph shows tumor area per slide level. F: Tumor volumes in cm³ of pio-treated animals compared to vehicle-treated animals. Data are presented as mean±SE of five animals per group (3 d, 6 d, 9 d, 14 d) or six animals per group (21 d). Asterisks indicate significant level in student-T-test: ***p<0 001 (n=5), **p<0 005 (n=5 or n=6). G: After 21 d of intracerebral pio treatment, tumor volumes of the C6 glioma model are reduced by 83% compared to vehicle treated animals. H: In the intracerebral treated group the tumor volumes are reduced by 76.9%. Data are presented as mean±SE of six animals per group. Asterisks indicate significant level in student-T-test: *p<0.05, **p<0 001 (n=6).

FIG. 3 illustrates pioglitazone treatment induced Bax- and cleaved caspase-3-upregulation in vivo. A: Bax and cleaved caspase-3 expression in whole-hemisphere lysates of C6 rat glioma animals and control at 3, 6, 9, and 14 d of treatment. Vehicle: vehicle-treated animals; Pio: pio-treated animals. Erk2 served as a loading control. B, C: Bax and cleaved caspase-3 expression at 6 d in 4 (vehicle) and 5 (pio) different animals. D, E: Densitometric analysis of Bax or cleaved caspase-3 levels at 3, 6, 9, 14 d: Data are presented as mean±SE of 3 different western blots. Black bars: vehicle treatment, gray bars: pio treatment. Asterisks indicate significant level in student-T-test: *p≦0 03 (n=3)**p≦0 002 (n=3), ***p≦0 001

FIG. 4 illustrates pioglitazone induced apoptosis and reduced proliferation. Cleaved caspase3 expression and TUNEL expression in the tumors of C6 rat glioma model animals with and without treatment at day 9 (n=5). DAPI counter staining to determine number of tumor cells. Bar: 100 μm. Percentage of cleaved caspase-3 positive tumor cells at 6, 9, and 14 d and TUNEL positive tumor cells at 9 d. Data are presented as mean±SE of the 5 animals per group. Black bars: vehicle treatment, gray bars: pio treatment. Asterisks indicate significant level in student-T-test: **p<0.008 (n=5). Ki-67 expression and BrdU incorporation in the tumors of C6 rat glioma model animals with and without treatment at day 14 (n=5). DAPI counter staining to determine number of tumor cells. Bar: 100 μm. Percentage of Ki-67 positive tumor cells (proliferation-index) at 9, 14, and 21 d and percentage of BrdU positive tumor cells at 14 days. Data are presented as mean±SE of the 5 animals per group. Black bars: vehicle treatment, gray bars: pio treatment. Asterisks indicate significant level in student-T-test: *p=0 0114 (n=5); **p≦0 006 (n=5 or n=5).

FIG. 5 illustrates pioglitazone reduces MMP9 protein levels and induces the astrocytic redifferentiation marker CS-56 in vivo. MMP-9 and CS-56 expression. A: MMP-9 expression in whole-hemisphere lysates of C6 rat glioma model animals using a goat-anti-MMP-9-antibody. Vehicle: vehicle-treated animals (n=3); Pio: pio-treated animals (n=3). To ensure equal loading of proteins, each membrane was stripped and reprobed with a rabbit anti-Erk2-antibody. B: Densitometry: Data are presented as mean±SE of the seven animals per group. Black bars: vehicle treatment, gray bars: pio treatment. Asterisks indicate significant level in student-T-test: ***p=0 0009 (n=3). C: CS-56 (red)/MMP-9 (green) expression at the tumor margin in brain tissue of vehicle- (two left rows) and pio- (two right rows) treated animals. Bars: 100 μm. DAPI-staining served as counterstaining. D: CS-56 expression in whole-hemisphere lysates of C6 rat glioma model animals using a mouse-anti-CS-56-antibody. Vehicle: vehicle-treated animals (n=3); Pio: pio-treated animals. To ensure equal loading of proteins, each membrane was stripped and reprobed with a rabbit anti-Erk2-antibody. E: Densitometry: Data are presented as mean±SE of the seven animals per group. Black bars: vehicle treatment, gray bars: pio treatment. Asterisks indicate significant level in student-T-test: **p=0.0012 (n=3).

### DETAILED DESCRIPTION

As used herein, the term “therapeutically effective amount” refers to that amount of a composition that results in amelioration of symptoms or a prolongation of survival in a patient. A therapeutically relevant effect relieves to some extent one or more symptoms of a disease or condition or returns to normal either partially or completely one or more physiological or biochemical parameters associated with or causative of the disease or condition.

As used herein, the term “PPARγ agonist” refers to a compound or composition, which when combined with PPARγ, directly or indirectly stimulates or increases an in vivo or in vitro reaction typical for the receptor (e.g., transcriptional regulation activity). The increased reaction can be measured by any of a variety of assays known to those skilled in the art. An example of a PPARγ agonist is a thiazolidinedione compound, such as troglitazone, rosiglitazone, pioglitazone, ciglitazone, WAY-120,744, englitazone, AD 5075, darglitazone, and congeners, analogs, derivatives, and pharmaceutically acceptable salts thereof.

As used herein, the terms “host” and “subject” refer to any animal, including, but not limited to, humans and non-human animals (e.g., rodents, arthropods, insects, fish (e.g., zebrafish), non-human primates, ovines, bovines, ruminants, lagomorphs, porcines, caprines, equines, canines, felines, aves, etc.), which is to be the recipient of a particular treatment. Typically, the terms “host,” “patient,” and “subject” are used interchangeably herein in reference to a human subject.

The term “biologically active,” as used herein, refers to a protein or other biologically active molecules (e.g., catalytic RNA) having structural, regulatory, or biochemical functions of a naturally occurring molecule.

The term “agonist,” as used herein, refers to a molecule which, when interacting with a biologically active molecule, causes a change (e.g., enhancement) in the biologically active molecule, which modulates the activity of the biologically active molecule. Agonists include, but are not limited to proteins, nucleic acids, carbohydrates, lipids or any other molecules which bind or interact with biologically active molecules. For example, agonists can alter the activity of gene transcription by interacting with RNA polymerase directly or through a transcription factor or signal transduction pathway.

The term “modulate,” as used herein, refers to a change in the biological activity of a biologically active molecule. Modulation can be an increase or a decrease in activity, a change in binding characteristics, or any other change in the biological, functional, or immunological properties of biologically active molecules.

As used herein, the term “in vitro” refers to an artificial environment and to processes or reactions that occur within an artificial environment. In vitro environments consist of, but are not limited to, test tubes and cell culture. The term “in vivo” refers to the natural environment (e.g., an animal or a cell) and to processes or reaction that occur within a natural environment.

“Treating” or “treatment” of a condition or disease includes: (1) preventing at least one symptom of the conditions, i.e., causing a clinical symptom to not significantly develop in a mammal that may be exposed to or predisposed to the disease but does not yet experience or display symptoms of the disease, (2) inhibiting the disease, i.e., arresting or reducing the development of the disease or its symptoms, or (3) relieving the disease, i.e., causing regression of the disease or its clinical symptoms. Treatment, prevention and ameliorating a condition, as used herein, can include, for example decreasing the volume of a tumor (e.g., glioma) and/or reducing Ki-67 expression.

The compositions and methods of the present invention are based on the use of PPARγ agonists to suppress, inhibit, modulate, and/or mitigate central nervous system tumor proliferation, growth, migration and/or metastasization, as shown by reduced tumor volume and/or size. Additionally, the compositions and methods of the present invention use PPARγ agonists to induce apoptotic cell death in central nervous system tumor cells in the subject's brain.

It was found that PPARγ agonists, such as a thiazolidinedione (e.g., pioglitazone), can be administered to a tumor (e.g., glioma) in the subject's brain to arrest the growth and invasion of the tumor (e.g., glioma) cells. Glioma proliferation was inhibited by PPARγ agonist treatment as evidenced by the reduction in expression of the proliferation marker Ki-67. Importantly, PPARγ agonist treatment resulted in the apoptotic death of glioma cells and also induced glioma redifferentiation as evidenced by increased CS-56 expression. It is of significance that PPARγ agonists acted to block glioma migration. Furthermore, non-thiazolidinedione PPARγ agonists also display anti-neoplastic effects in glioma cell lines.

Additionally, it was found that continuous intracerebral infusion of a PPARγ agonist (e.g., pioglitazone) into gliomas, induced by intrastriatal injection of C6 glioma cells into the brain, reduced tumor volumes by 83%. Oral administration of the PPARγ agonist reduced tumor volumes by 77%. Subsequent analysis revealed a drug-related suppression of glioma proliferation, as measured by BrdU and Ki-67 labeling, and induction of apoptotic cell death that was restricted to the neoplastic cells. PPARγ agonist treatment resulted in apoptosis of the glioma cells, as evidenced by detection of cleaved forms of caspase 3, TUNEL staining and induction of Bax expression. Importantly, treatment of the brain tumors in vivo with a PPARγ agonist dramatically reduced well defined tumor margins and dramatically reduced invasion of glioma cells into the adjacent brain tissue. The reduced invasiveness was verified using a Boyden chamber assay, which demonstrated that PPARγ agonist treatment reduced the capacity of glioma cells to migrate. This was associated with drug-induced reduction in brain MMP9 levels, a proteinase which is important in glioma invasion and a direct target of PPARγ action and stimulated the redifferentiation of the glioma cells, as monitored by expression of CS-56. Importantly, PPARγ agonist therapy resulted in a dramatic improvement in clinical outcomes, with drug treated animals exhibiting less hemiparesis, no cycling, and less immobility than vehicle treated control. These data provide critical proof of concept evidence for the utility of PPARγ agonist in treatment of central nervous system tumors.

One aspect of the present invention therefore relates to method of treating central nervous system tumors in a subject by administering a therapeutically effective amount of compounds that include PPARγ agonists or therapeutically effective derivatives thereof to the tumor in the subject.

The central nervous system tumor can include, for example, brain tumors, gliomas, glioblastoma multiforme, neuroblastomas, medullablastomas as well as other very aggressive central nervous system tumors. In an aspect of the invention, the central nervous system tumor can include a glioma of the subject's brain.

The PPARγ agonists administered to the tumor can include, for example, prostaglandin J2 (PGJ2) and analogs thereof (e.g., A2-prostaglandin J2 and 15-deoxy-2 4-prostaglandin J2), members of the prostaglandin D2 family of compounds, docosahexaenoic acid (DHA), and thiazolidinediones (e.g., ciglitazone, troglitazone, pioglitazone, and rosiglitazone).

In addition, such agents include, but are not limited to, L-tyrosine-based compounds, farglitazar, GW7845, indole-derived compounds, indole 5-carboxylic acid derivatives and 2,3-disubstituted indole 5-phenylacetic acid derivatives. It is significant that most of the PPARγ agonists exhibit substantial bioavailability following oral administration and have little or no toxicity associated with their use (See e.g., Saltiel and Olefsky, Diabetes 45:1661 (1996); Wang et al, Br. J. Pharmacol. 122:1405 (1997); and Oakes et al, Metabolism 46:935 (1997)). It will be appreciated that the present invention is not limited to above-identified PPARγ agonists and that other identified PPARγ agonists can also be used. PPARγ agonists that can be used for practicing the present invention, and methods of making these compounds are disclosed in WO 91/07107; WO 92/02520; WO 94/01433; WO 89/08651; WO 96/33724; WO 97/31907; U.S. Pat. Nos. 4,287,200; 4,340,605; 4,438,141; 4,444,779; 4,461,902; 4,572,912; 4,687,777; 4,703,052; 4,725,610; 4,873,255; 4,897,393; 4,897,405; 4,918,091; 4,948,900; 5,002,953; 5,061,717; 5,120,754; 5,132,317; 5,194,443; 5,223,522; 5,232,925; 5,260,445; 5,814,647; 5,902,726; 5,994,554; 6,294,580; 6,306,854; 6,498,174; 6,506,781; 6,541,492; 6,552,055; 6,579,893; 6,586,455, 6,660,716, 6,673,823; 6,680,387; 6,768,008; 6,787,551; 6,849,741; 6,878,749; 6,958,355; 6,960,604; 7,022,722 and U.S. Applications 20030130306, 20030134885, 20030109579, 20030109560, 20030088103, 20030087902, 20030096846, 20030092697, 20030087935, 20030082631, 2003007g288, 20030073862, 20030055265, 20030045553, 1 20020169192, 20020165282, 20020160997, 20020128260, 20020103188, 20020082292, 20030092736, 20030069275, 20020151569, and 20030064935.

The disclosures of these publications are incorporated herein by reference in their entireties, especially with respect to the PPARγ agonists disclosed therein, which may be employed in the methods described herein.

As PPARγ agonist having the aforementioned effects, the compounds of the following formulas are useful in treating individuals. Accordingly, in some embodiments of the present invention, the therapeutic agents comprise compounds of

### Formula I:

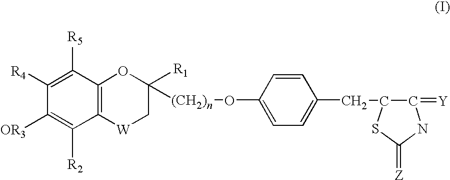

wherein R₁ and R₂ are the same or different, and each represents a hydrogen atom or a C₁-C₅ alkyl group; R₃ represents a hydrogen atom, a C₁-C₆ aliphatic acyl group, an alicyclic acyl group, an aromatic acyl group, a heterocyclic acyl group, an araliphatic acyl group, a (C₁-C₆ alkoxy)carbonyl group, or an aralkyloxycarbonyl group; R₄ and R₅ are the same or different, and each represents a hydrogen atom, a C₁-C₅ alkyl group or a C₁-C₅ alkoxy group, or R₄ and R₅ together represent a C₁-C₅ alkylenedioxy group; n is 1, 2, or 3; W represents the CH₂, CO, or CHOR₆ group (in which R₆ represents any one of the atoms or groups defined for R₃ and may be the same as or different, from R₃); and Y and Z are the same or different and each represents an oxygen atom or an imino (—NH) group; and pharmaceutically acceptable salts thereof.

In some embodiments of the present invention, the PPARγ agonist comprise compounds of Formula II:

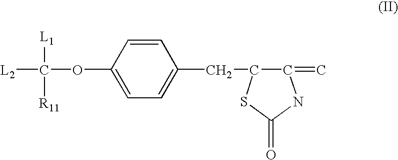

wherein R₁₁, is a substituted or unsubstituted alkyl, alkoxy, cycloalkyl, phenylalkyl, phenyl, aromatic acyl group, a 5- or 6 membered heterocyclic group including 1 or 2 heteroatoms selected from the group consisting of nitrogen, oxygen, and sulfur, or a group of the formula indicated in:

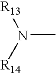

wherein R₁₃ and R₁₄ are the same or different and each is a lower alkyl (alternately, R₁₃ and R₁₄ are combined to each other either directly or as interrupted by a heteroatom comprising nitrogen, oxygen, and sulfur to form a 5- or 6-membered ring); and wherein L¹ and L² are the same or different and each is hydrogen or lower alkyl or L¹ and L² are combined to form an alkylene group; or a pharmaceutically acceptable salt thereof.

In some aspects of the present invention, the therapeutic agents comprise compounds of Formula III:

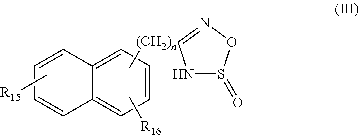

wherein R₁₅ and R₁₆ are independently hydrogen, lower alkyl containing 1 to 6 carbon atoms, alkoxy containing 1 to 6 carbon atoms, halogen, ethyl, nitrite, methylthio, trifluoromethyl, vinyl, nitro, or halogen substituted benzyloxy; n is 0 to 4; or a pharmaceutically acceptable salt thereof.

In some aspects of the present invention, the PPARγ agonist comprise compounds of Formula IV:

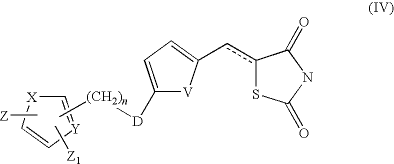

wherein the dotted line represents a bond or no bond; V is HCH—, —NCH—, —CH═N—, or S; D is CH₂, CHOH, CO, C═NOR₁₇, or CH═CH; X is S, SO, NR₁₈, —CH═N, or —N═CH; Y is CH or N; Z is hydrogen, (C₁-C₇)alkyl, (C₁-C₇)cycloalkyl, phenyl, naphthyl, pyridyl, furyl, thienyl, or phenyl mono- or di-substituted with the same or different groups which are (C₁-C₃)alkyl, trifluoromethyl, (C₁-C₃)alkoxy, fluoro, chloro, or bromo; Z₁ is hydrogen or (C₁-C₃)alkyl; R₁₇ and R₁₈ are each independently hydrogen or methyl; and n is 1, 2, or 3; the pharmaceutically acceptable cationic salts thereof; and the pharmaceutically acceptable acid addition salts thereof when the compound contains a basic nitrogen.

In some embodiments of the present invention, the PPARγ agonist comprise compounds of Formula V:

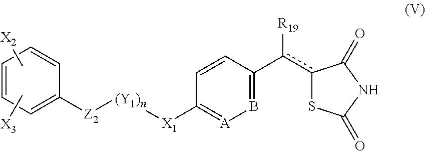

wherein the dotted line represents a bond or no bond; A and B are each independently CH or N. with the proviso that when A or B is N. the other is CH; X is S, SO, SO₂, CH₂, CHOH, or CO; n is 0 or 1; Y₁ is CHR₂₀ or R₂₁, with the proviso that when n is 1 and Y₁ is NR₂₁, X₁ is SO₂ or CO; Z₂ is CHR₂₂, CH₂CH₂, cyclic C₂H₂O, CH═CH, OCH₂, SCH₂, SOCH₂, or SO₂CH₂; R₁₉, R₂₀, R₂₁, and R₂₂ are each independently hydrogen or methyl; and X₂ and X₃ are each independently hydrogen, methyl, trifluoromethyl, phenyl, benzyl, hydroxy, methoxy, phenoxy, benzyloxy, bromo, chloro, or fluoro; a pharmaceutically acceptable cationic salt thereof; or a pharmaceutically acceptable acid addition salt thereof when A or B is N.

In some embodiments of the present invention, the PPARγ agonist comprise compounds of Formula VI:

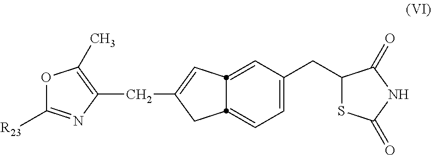

or a pharmaceutically acceptable salt thereof, wherein R₂₃ is alkyl of 1 to 6 carbon atoms, cycloalkyl of 3 to 7 carbon atoms, phenyl or mono- or all-substituted phenyl wherein said substituents are independently alkyl of 1 to 6 carbon atoms, alkoxy of 1 to 3 carbon atoms, halogen, or trifluoromethyl.

In some embodiments of the present invention, the PPARγ agonist comprise compounds of Formula VII:

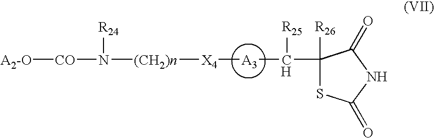

or a tautomeric form thereof and/or a pharmaceutically acceptable salt thereof, and/or a pharmaceutically acceptable solvate thereof, wherein: A₂ represents an alkyl group, a substituted or unsubstituted aryl group, or an aralkyl group wherein the alkylene or the aryl moiety may be substituted or unsubstituted; A³ represents a benzene ring having in total up to 3 optional substituents; R₂₄ represents a hydrogen atom, an alkyl group, an acyl group, an aralkyl group wherein the alkcyl or the aryl moiety may be substituted or unsubstituted, or a substituted or unsubstituted aryl group; or A₂ together with R₂₄ represents substituted or unsubstituted C₂₋₃ polymethylene group, optional substituents for the polymethylene group being selected from alkyl or aryl or adjacent substituents together with the methylene carbon atoms to which they are attached form a substituted or unsubstituted phenylene group; R₂₅ and R₂₆ each represent hydrogen, or R₂₅ and R₂₆ together represent a bond; X₄ represents O or S; and n represents an integer in the range from 2 to 6.

In some embodiments of the present invention, the PPARγ agonists comprise compounds of Formula VIII:

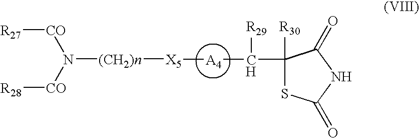

or a tautomeric form thereof and/or a pharmaceutically acceptable salt thereof, and/or a pharmaceutically acceptable solvate thereof, wherein: R₂₇ and R₂₈ each independently represent an alkyl group, a substituted or unsubstituted aryl group, or an aralkyl group being substituted or unsubstituted in the aryl or alkyl moiety; or R₂₇ together with R₂₈ represents a linking group, the linking group consisting or an optionally substituted methylene group or an O or S atom, optional substituents for the methylene groups including alkyl, aryl, or aralkyl, or substituents of adjacent methylene groups together with the carbon atoms to which they are attached form a substituted or unsubstituted phenylene group; R₂₉ and R₃₀ each represent hydrogen, or R₂₉ and R₃₀ together represent a bond; A₄ represents a benzene ring having in total up to 3 optional substituents; X₅ represents O or S; and n represents an integer in the range of 2 to 6.

In some embodiments of the present invention, the PPARγ agonist comprise compounds of Formula IX:

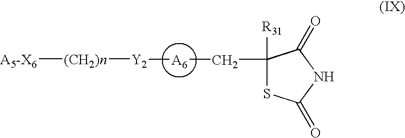

or a tautomeric form thereof and/or a pharmaceutically acceptable salt thereof, and/or a pharmaceutically acceptable solvate thereof, wherein: A₅ represents a substituted or unsubstituted aromatic heterocyclyl group; A₆ represents a benzene ring having in total up to 5 substituents; X₆ represents O, S, or NR₃₂ wherein R₃₂ represents a hydrogen atom, an alkyl group, an acyl group, an aralkyl group, wherein the aryl moiety may be substituted or unsubstituted, or a substituted or unsubstituted aryl group; Y₂ represents O or S; R₃₁ represents an alkyl, aralkyl, or aryl group; and n represents an integer in the range from 2 to 6. Aromatic heterocyclyl groups include substituted or unsubstituted, single or fused ring aromatic heterocyclyl groups comprising up to 4 hetero atoms in each ring selected from oxygen, sulfur, or nitrogen. Aromatic heterocyclyl groups include substituted or unsubstituted single ring aromatic heterocyclyl groups having 4 to 7 ring atoms, preferably 5 or 6 ring atoms.

In particular, the aromatic heterocyclyl group comprises 1, 2, or 3 heteroatoms, especially 1 or 2, selected from oxygen, sulfur, or nitrogen. Values for A₅ when it represents a 5-membered aromatic heterocyclyl group include thiazolyl and oxazoyl, especially oxazoyl. Values for A₆ when it represents a 6 membered aromatic heterocyclyl group include pyridyl or pyrimidinyl. R₃₁ represents an alkyl group, in particular a C-6 allcyl group (e.g., a methyl group).

A⁵ can represent a moiety of formula (a), (b), or (c), under Formula IX:

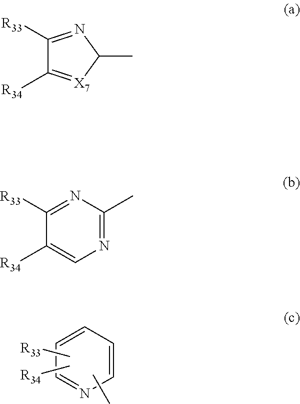

wherein, R₃₃ and R₃₄ each independently represents a hydrogen atom, an alkyl group, or a substituted or unsubstituted aryl group or when R₃₃ and R₃₄ are each attached to adjacent carbon atoms, then R₃₃ and R₃₄ together with the carbon atoms to which they are attached forth a benzene ring wherein each carbon atom represented by R₃₃ and R₃₄ together may be substituted or unsubstituted; and in the moiety of Formula (a), X₇ represents oxygen or sulphur.

In one embodiment of the present invention, R₃₃ and R₃₄ together present a moiety of Formula (d) in FIG. 8, under Formula IX:

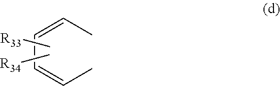

wherein R₃₅ and R₃₆ each independently represent hydrogen, halogen, substituted or unsubstituted alkyl, or alkoxy.

In some embodiments of the present invention, the PPARγ agonist comprise compounds of Formula X:

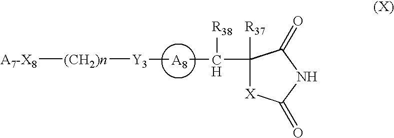

or a tautomeric form thereof and/or a pharmaceutically acceptable salt thereof, and/or a pharmaceutically acceptable solvate thereof, wherein: A₇ represents a substituted or unsubstituted aryl group; A₈ represents a benzene ring having in total up to 5 substituents; X₈ represents O, S, or NR₉, wherein R₃₉ represents a hydrogen atom, an alkyl group, an acyl group, an aralkyl group, wherein the aryl moiety may be substituted or unsubstituted, or a substituted or unsubstituted aryl group; Y₃ represents O or S; R₃₇ represents hydrogen; R₃₈ represents hydrogen or an alkyl, aralkyl, or aryl group or R₃₇ together with R₃₈ represents a bond; and n represents an integer in the range from 2 to 6.

In some embodiments of the present invention, the PPARγ agonist comprise compounds of Formula XI:

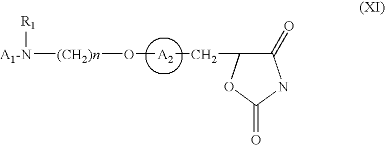

or a tautomeric form thereof and/or a pharmaceutically acceptable salt thereof, and/or a pharmaceutically acceptable solvate thereof, wherein: A₁ represents a substituted or unsubstituted aromatic heterocyclyl group; R₁ represents a hydrogen atom, an alkyl group, an acyl group, an aralkyl group, wherein the aryl moiety may be substituted or unsubstituted, or a substituted or unsubstituted aryl group; A₂ represents a benzene ring having in total up to 5 substituents; and n represents an integer in the range of from to 6. Suitable aromatic heterocyclyl groups include substituted or unsubstituted, single or fused ring aromatic heterocyclyl groups comprising up to 4 hetero atoms in each ring selected from oxygen, sulfur, or nitrogen. Favored aromatic heterocyclyl groups include substituted or unsubstituted single ring aromatic heterocyclyl groups having 4 to 7 ring atoms, preferably 5 or 6 ring atoms. In particular, the aromatic heterocyclyl group comprises 1, 2, or 3 heteroatoms, especially 1 or 2, selected from oxygen, sulfur, or nitrogen. Values for A₁ when it represents a 5-membered aromatic heterocyclyl group can include thiazolyl and oxazolyl, especially oxazoyl. Values for A₁ when it represents a 6-membered aromatic heterocyclyl group can include pyridyl or pyrimidinyl.

In some embodiments of the present invention, the PPARγ agonists comprise a compound of Formulas XII and XIII:

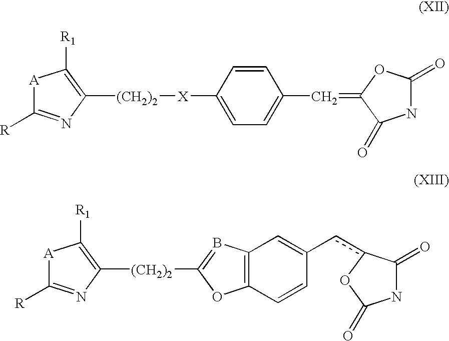

or pharmaceutically acceptable salts thereof wherein the dotted line represents a bond or no bond; R is cycloalkyl of three to seven carbon atoms, naphthyl, thienyl, furyl, phenyl, or substituted phenyl wherein the substituent is alkyl of one to three carbon atoms, alkoxy of one to three carbon atoms, trifluoromethyl, chloro, fluoro, or bis(trifluoromethyl); R₁ is an alkyl of one to three carbon atoms; X is O or C═O; A is O or S; and B is N or CH.

Some embodiments of the present invention include the use of the compounds of Formulas I through XIII are referred to as thiazolidine derivatives. Where appropriate, the specific names of thiazolidine derivatives may be used including: troglitazone, ciglitazone, pioglitazone, and rosiglitazone.

In certain embodiments, the therapeutic agent comprises an activator of PPARγ as described in U.S. Pat. No. 5,994,554, e.g., having a structure selected from the group consisting of formulas (XIV)-(XXVI):

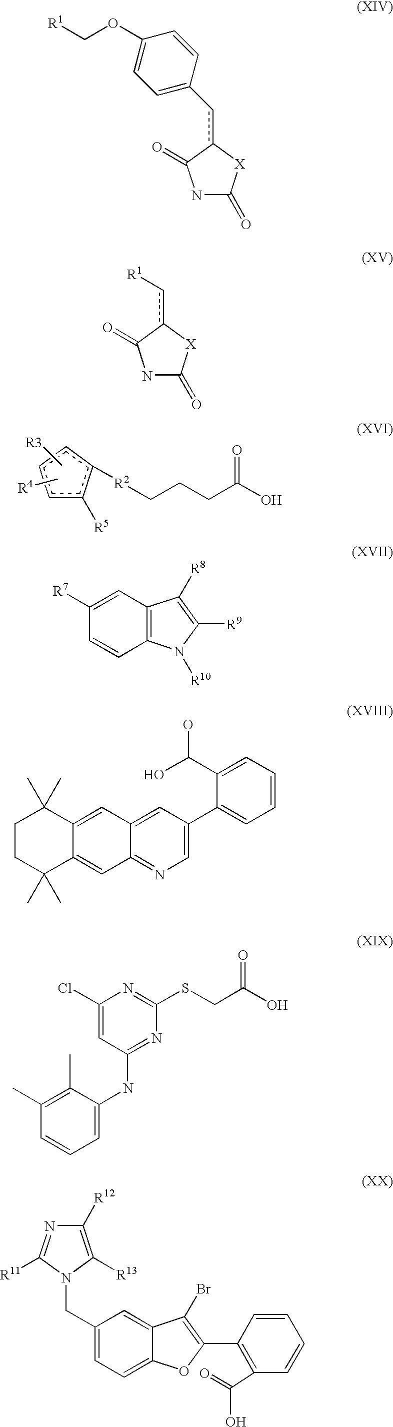

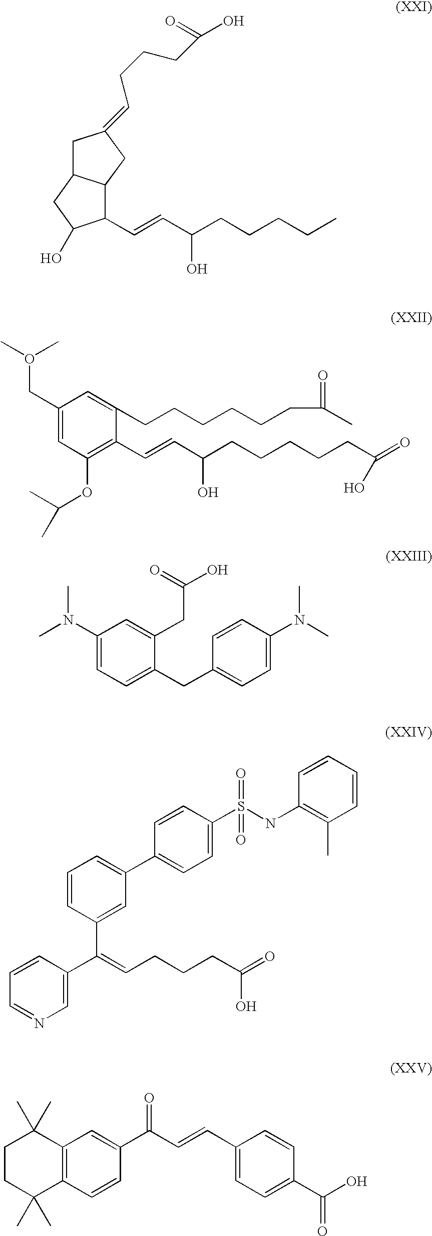

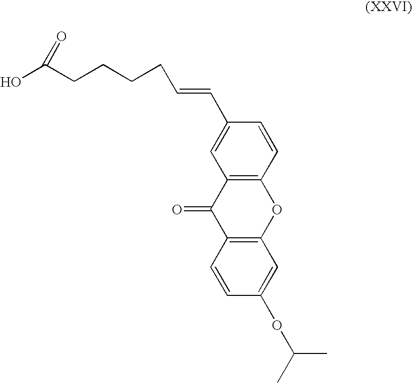

wherein: R¹ is selected from the group consisting of hydrogen, C₁₋₈ alkyl, aminoC₁₋₈, alkyl, C₁₋₈alkylamino C₁₋₈ alkyl, heteroarylamino C₁₋₆ alkyl, (heteroaryl)(C₁₋₈alkyl)aminoC₁₋₆ alkyl, (C₁₋₈ cycloalkyl) C₁₋₈ alkyl, C₁₋₈ alkylheteroaryl C₁₋₈ alkyl, 9- or 10-membered heterobicycle, which is partially aromatic or substituted 9- or 10-membered heterobicycle, which is partially aromatic; X is selected from the group consisting of S, NH, or O; R² is selected from the group consisting of hydrogen, C₁₋₈allcyl or C₁₋₈alkenyl; R³ and R⁴ are independently selected from the group consisting of hydrogen, hydroxy, oxo C₁₋₈alkyl, C₁₋₈alkoxy or amino; R⁵ is selected from the group consisting of hydrogen, C₁₋₈alkyl, C₁₋₈alkenyl, (carbonyl)alkenyl, (hydroxy)alkenyl, phenyl, C₁₋₈alkylR⁶, (hydroxy) C₁₋₈alkylR⁶, C₁₋₈alkyl C₁₋₈cycloallcylR⁶, (hydroxy) C₁₋C₁₋₈cycloallcylR⁶ or C₁₋₈cycloallcylthioR⁶; R⁶ is selected from the group consisting of phenyl or phenyl substituted with hydroxy, C₁₋₈alkyl or C₁₋₈alkoxy substituents; R⁷ is selected from the group consisting of hydrogen, hydroxy, carboxy or carboxy C₁₋₈alkyl; R⁸ is selected from the group consisting of hydrogen, C₁₋₈alkyl, phenyl, phenyl C₁₋₈alkyl, phenyl mono- or all-substituted with halo, hydroxy, and/or C₁₋₈alkoxy (e.g., methoxy) substituents or phenyl C₁₋₈alkyl wherein the phenyl is mono- or disubstituted with halo, hydroxy, and/or C₁₋₈alkoxy (e.g., methoxy) substituents; R⁹ is selected from the group consisting of hydrogen, C₁₋₈alkyl, carboxy C₁₋₈alkenyl mono- or disubstituted with hydroxy, and/or C₁₋₈alkoxy (e.g., methoxy), phenyl or phenyl mono- or disubstituted with halo, hydroxy, and/or C₁₋₈alkoxy (e.g., methoxy) R¹⁰ is hydrogen or C₁-8alkyl, R¹¹ is selected from the group consisting of hydrogen, C₁₋₈alkyl or cycloC₁₋₈alkyl C₁₋₈alkyl; R¹² is selected from the group consisting of hydrogen, halo or fluorinated C₁₋₈alkyl; R¹³ is selected from the group consisting of hydrogen, C₁₋₈alkoxycarbonyl or C₁₋₈alkoxycarbonyl C₁₋₈alkylaminocarbonyl; a dashed line ( - - - ) is none or one double bond between two of the carbon atoms; fluorinated alkyl can be an alkyl wherein one or more of the hydrogen atoms is replaced by a fluorine atom; heteroaryl can be 5, 6 or 7 membered aromatic ring optionally interrupted by 1, 2, 3 or 4 N, S, or O heteroatoms, with the proviso that any two O or S atoms are not bonded to each other; substituted heteroaryl can be a 9- or 10-membered heterobicycle mono-, di-, or trisubstituted independently with hydroxy, oxo, C₁₋₆ alkyl, C₁₋₆ alkoxy or 9- or 10-membered heterobicycle, which is partially aromatic in more detail is a heterobicycle interrupted by 1, 2, 3, or 4 N heteroatoms; substituted 9- or 10-membered heterobicycle, which is partially aromatic in more detail is a 9- or 10-membered heterobicycle mono-, di-, tri- or tetrasubstituted independently with hydroxy, oxo, C₁₋₈ alkyl, C₁₋₈ alkoxy, phenyl, phenyl C₁₋₈ alkyl; or a pharmaceutically acceptable acid-addition or base-addition salt thereof.

In yet other embodiments, the PPARγ agonist comprises a compound as disclosed in U.S. Pat. No. 6,306,854, e.g., a compound having a structure of Formula (XXVII):

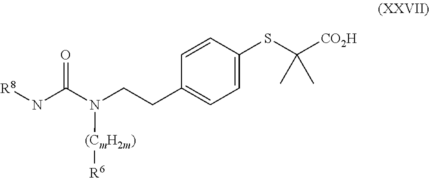

and esters, salts, and physiologically functional derivatives thereof; wherein m is from 0 to 20, R⁶ is selected from the group consisting of hydrogen and

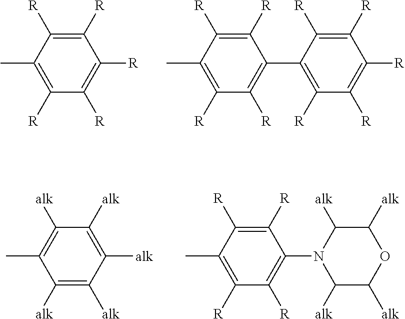

and R⁸ is selected frown the group consisting of:

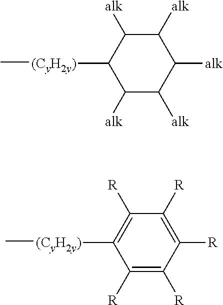

where y is 0, 1, or 2, each alk is independently hydrogen or alkyl group containing 1 to 6 carbon atoms, each R group is independently hydrogen, halogen, cyano, —NO₂, phenyl, straight or branched alkyl or fluoroalkyl containing 1 to 6 carbon atoms and which can contain hetero atoms such as nitrogen, oxygen, or sulfur and which can contain functional groups such as ketone or ester, cycloalkyl containing 3 to 7 carbon atoms, or two R groups bonded to adjacent carbon atoms can, together with the carbon atoms to which they are bonded, form an aliphatic or aromatic ring or multi ring system, and where each depicted ring has no more than 3 alk groups or R groups that are not hydrogen.

In yet other embodiments of the present invention a PPARγ agonist is a compound such as disclosed in U.S. Pat. No. 6,294,580 and/or Liu et al., Biorg. Med. Chem. Lett. 11 (2001) 3111-3113, e.g., having a structure within Formula XXVIII:

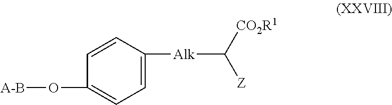

wherein A is selected from the group consisting of: (i) phenyl, wherein said phenyl is optionally substituted by one or more of the following groups; halogen atoms, C₁₋₆alkyl, C₁₋₃ alkoxy, C₁₋₃ fluoroalkoxy, nitrite, or —NR⁷R⁸ where R⁷ and R⁸ are independently hydrogen or C₁₋₃ alkyl; (ii) a 5- or 6-membered heterocyclic group containing at least one heteroatom selected from oxygen, nitrogen and sulfur; and (iii) a fused bicyclic ring

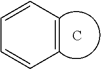

wherein ring C represents a heterocyclic group as defined in point (ii) above, which bicyclic ring is attached to group B via a ring atom of ring C; B is selected from the group consisting of: (iv) C₁₋₆ alkylene; (v) -M C₁₋₆ alkylene or C₁₋₆ alkyleneM C₁₋₆ alkylene, wherein M is O, S, or —NR² wherein R² represents hydrogen or C₁₋₃ alkyl; (vi) a 5- or 6-membered heterocyclic group containing at least one nitrogen heteroatom and optionally at least one further heteroatom selected from oxygen, nitrogen and sulfur and optionally substituted by C₁₋₃ alkyl; and (vii) Het-C₁₋₆ alkylene, wherein Het represents a heterocyclic group as defined in point (vi) above; Alk represents C₁₋₃ alkylene; Het represents hydrogen or C₁₋₃ alkyl; Z is selected from the group consisting of: (viii) nitrogen-containing heterocyclyl or heteroaryl, e.g., N-pyrrolyl, N-piperidinyl, N-piperazinyl, N-morpholinyl, or N-imidazolyl, optionally substituted with 1-4 C₁₋₆ alkyl or halogen substituents; (ix) —(C₁₋₃ alkylene)phenyl, which phenyl is optionally substituted by one or more halogen atoms; and (x) —NR³R⁴, wherein R³ represents hydrogen or C₁₋₃ alkyl, and R⁴ represents C₁₋₆ alkyl, aryl or heteroaryl (e.g., phenyl, pyridinyl, pyrazinyl, pyrimidinyl, pyrrolyl, piperidinyl, piperazinyl, morpholinyl, imidazolyl), optionally substituted by 1-4 C₁₋₆ alkyl, halogen, C₁₋₆ alkoxyl, hydroxyl, nitro, cyano, or amino substituents, or —Y—(C═O)-T—R⁵, —Y—SO₂—R⁵, or —Y—(CH(OH))-T—R⁵, wherein: (a) Y represents a bond, C₁₋₆ alkylene, C₂₋₆ alkenylene, C₄₋₆ cycloalkylene or cycloalkenylene, a heterocyclic group as defined in point (vi) above, or phenyl optionally substituted by one or more C₁₋₃ alkyl groups and/or one or more halogen atoms; (b) T represents a bond, C₁₋₃ alkyleneoxy, —O— or —N(R⁶)—, wherein R⁵ represents hydrogen or C₁₋₃ alkyl; (c) R⁵ represents C₁₋₆ alkyl, C₄₋₆ cycloalkyl or cycloalkenyl, phenyl (optionally substituted by one or more of the following groups; halogen atoms, C₁₋₃ alkyl, C₁₋₃ alkoxy groups, C₁₋₃ alkyleneNR⁹, R¹⁰ (where each R⁹ and R¹⁰ is independently hydrogen, C₁₋₃ alkyl, —SO₂C₁₋₃ alkyl, or —CO₂C₁₋₃ alkyl, —SO₂ NHC₁₋₃ alkyl), C₁₋₃ alkyleneCO₂H, C₁₋₃alkyleneCO₂C₁₋₃ alkyl, or —OCH₂C(O)NH₂), a 5- or 6 membered heterocyclic group as defined in point (ii) above, a bicylic fused ring

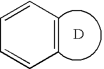

wherein ring D represents a 5- or 6-membered heterocyclic group containing at least one heteroatom selected from oxygen, nitrogen and sulfur and optionally substituted by (═O), which bicyclic ring is attached to T via a ring atom of ring D: or —C₁₋₆ alkyleneMR¹¹ M is O, S, or —NR¹² wherein R¹¹ and R¹² are independently hydrogen or C₁₋₃ alkyl, or a tautomeric form thereof, and/or a pharmaceutically acceptable salt or solvate thereof.

One specific group of compounds are those of Formula XI, wherein the dotted line represents no bond, R¹ is methyl, X is O and A is O. Examples of compounds in this group are those compounds where R is phenyl, 2-naphthyl and 3,5bis(trifluoronethyl)phenyl. Another specific group of compounds are those of Formula XIII, wherein the dotted line represents no bond, R¹ is methyl and A is O. Particularly preferred compounds within this group are compounds where B is CH and R is phenol, p-tolyl, m-tolyl, cyclohexyl, and 2-naphthyl. In alternative embodiments of the present invention, the B is N and R is phenyl.

In still further embodiments, the present invention provides methods for the use of a pharmaceutical composition suitable for administering an effective amount of at least one composition comprising a PPARγ agonist, such as those disclosed herein, in unit dosage form to treat reperfusion related injury associated with reperfusion of ischemic tissue following a cerebrovascular accident. In alternative embodiments, the composition further comprises a pharmaceutically acceptable carrier.

Specific examples of compounds of the present invention are given in the following list: (+)-5 [[4-[(3,4-dihydro-6-hydroxy-2,5,7,8-tetramethyl-2H-1-benzopyran-2-yl)methoxy]phenyl]methyl]-2,4thiazolidinedione; (Troglitazone); 5-[4-[2-(5-ethylpyridin-2-yl)ethoxyl]benzyl]thiazolidine-2,4-dione; (pioglitazone); 5-[4-[(1-methylcyclohexyl)methoxy]benzyl]thiazolidine-2,4-dione; (ciglitazone); 4-(2-naphthylmethyl)-1,2,3,5-oxathiadiazole-2-oxide; 5-[4-[2-[(N-(benzoxazol-2-yl)-N-methylamino]ethoxy]benzyl]-5-methlthiazolidine-2,4-dione; 5-[4-[2-[2,4dioxo-5-phenylthiazolidine-3-yl)ethoxy]benzyl]thiazolidine-2,4-dione; 5-[4-[2-[(N-methyl-N-(phenoxycarbonyl)amino]ethoxy]benzyl]thiazolidine-2,4-dione; 5-[4-[2-phenoxyethoxy)benzyl]thiazolidine-2,4-dione; 5-[4-[2-(4-chorophenyl)ethylsulfonyl]benzyl]thiazolidine-2,4-dione; 5-[4-[3-(5-methyl-2-phenyloxazol-4-yl)propionyl]benzyl]thiazolidine-2,4-dione; 5-[[4-(3-hydroxy-1-methylcyclohexyl)methoxy]benzyl]thiazolidine-2,4-dione; 5-[4-[2-(5-methyl-2-phenyloxazol-4-yl)ethoxyl]benzyl]thiazolidine-2,4-dione; 5-[(2-benzyl-2,3-dihydrobenzopyran)-5-ylmethyl]thiazolidine-2,4-dione; (englitazone); 5-[[2-(2-naphthylmethyl)benzoxazol]-5-ylmethyl]thiazolidine-2,4-dione; 5-[4-[2-(3-phenylureido)ethoxyl]benzyl]thiazolidine-2,4-dione; 5-[4-[2-(N-benzoxazol-2-yl)-N-metholamino]ethoxy]benzyl]thiazolidine-2,4-dione; 5-[4-[3-(5-methyl-2-phenyloxazol-4-yl)propionyl]benzyl]thiazolidine-2,4-dione; 5-[2-(5-methyl-2-phenyloxazol-4-ylmethyl)benzofuran-5-ylmethyl]oxazolidine-2,4-dione; 5-[4-[2-(N-methyl-N-(2-pyridyl)amino]ethoxy]benzyl]thiazolidine-2,4-dione (rosiglitazone); and 5-[4-[2-(N-(benzoxazol-2-yl)-N-methylamino]ethoxy]benzyl]oxazolidine-2,4-di-one.

In yet other embodiments of the present invention, the therapeutic agents comprise compounds having the structure shown in Formula XXIX:

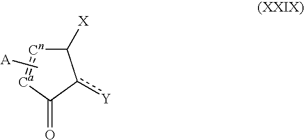

wherein: A is selected from hydrogen or a leaving group at the α- or β-position of the ring, or A is absent when there is a double bond between the Ca and Cn of the ring; X is an alkyl, substituted alkyl, alkenyl, substituted alkenyl, alkynyl, or substituted alkynyl group having in the range of 2 up to 15 carbon atoms; and Y is an alkyl, substituted alkyl, alkenyl, substituted alkenyl, alkynyl, or substituted alkynyl group having in the range of 2 up to 15 carbon atoms. As used herein, the term “leaving group” refers to functional groups which can readily be removed from the precursor compound, for example, by nucleophilic displacement, under E2 elimination conditions, and the like. Examples include, but are limited to, hydroxy groups, alkoxy groups, tosylates, brosylates, halogens, and the like.

The therapeutic agents of the present invention (e.g., the compounds in Formulas I-XXIX and the others described above) are capable of further forming both pharmaceutically acceptable acid addition and/or base salts. All of these forms are within the scope of the present invention and can be administered to the subject to treat gliomas.

Pharmaceutically acceptable acid addition salts of the present invention include, but are not limited to, salts derived from nontoxic inorganic acids such as hydrochloric, nitric, phosphoric, sulfuric, hydrobromic, hydriodic, hydrofluoric, phosphorous, and the like, as well as the salts derived forth nontoxic organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. Such salts thus include sulfate, pyrosulfate, bisulfate, sulfite, bisulfite, nitrate, phosphate, monoLydrogenphosphate, dihydrogenphosphate, metaphosphate, pyrophosphate, chloride, bromide, iodide, acetate, trifluoracetate, propionate, caprylate, isobutyrate, oxalate, malonate, succinate, suberate, sebacate, fumarate, malcate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Also contemplated are salts of amino acids such as arginate and the like, as well as gluconate, galacturonate, and n-methyl glucamine.

The acid addition salts of the basic compounds are prepared by contacting the free base form with a sufficient amount of the desired acid to produce the salt in the conventional manner. The free base form may be regenerated by contacting the salt form with a base and isolating the free base in the conventional manner or as described above. The free base forms differ from their respective salt forms somewhat in certain physical properties such as solubility in polar solvents, but are otherwise equivalent to their respective free base for purposes of the present invention.

Pharmaceutically acceptable base addition salts are formed with metals or amides, such as alkali and alkaline earth metals or organic amines. Examples of metals used as cations include, but are not limited to, sodium, potassium, magnesium, calcium, and the like. Examples of suitable amines include, but are not limited to, N₂—N′-dibenzylethylenediamine, chloroprocaine, choline, diethanolamine, dicyclohexylamine, ethylenediamine, N-methylglucamine, and procaine.

The base addition salts of the acidic compounds are prepared by contacting the free acid form with a sufficient amount of the desired base to produce the salt in the conventional manner. The free acid form may be regenerated by contacting the salt form with an acid and isolating the free acid in the conventional manner or as described above. The free acid forms differ from their respective salt forms somewhat in certain physical properties such as solubility in polar solvents, but otherwise the salts are equivalent to their respective free acid for purposes of the present invention.

Certain of the compounds of the present invention can exist in unsolvated forms as well as solvated forms, including, but not limited to, hydrated forms in general, the solvated forms, including hydrated forms, are equivalent to unsolvated forms and are intended to be encompassed within the scope of the present invention. Certain of the compounds of the present invention possess one or more chiral centers and each center may exist in different configurations. The compounds can, therefore, form stereoisomers. Although these are all represented herein by a limited number of molecular formulas, the present invention includes the use of both the individual, isolated isomers and mixtures, including racemates, thereof. Where stereospecific synthesis techniques are employed or optically active compounds are employed as starting materials in the preparation of the compounds, individual isomers may be prepared directly. However, if a mixture of isomers is prepared, the individual isomers may be obtained by conventional resolution techniques, or the mixture may be used as is, with resolution.

Furthermore, the thiazolidene or oxazolidene part of the compounds of Formulas I through XIII can exist in the form of tautomeric isomers, and are intended to be a part of the present invention.

For preparing pharmaceutical compositions from the compounds of the present invention, pharmaceutically acceptable carriers can be in any suitable form (e.g., solids, liquids, gels, etc.). Solid form preparations include, but are not limited to, powders, tablets, pills, capsules, cachets, suppositories, and dispersible granules. A solid carrier can be one or more substances which may also act as diluents, flavoring agents, binders, preservatives, tablet disintegrating agents, or an encapsulating material.

The present invention contemplates a variety of techniques for administration of the therapeutic compositions. Examples of routes include, but are not limited to, oral, rectal, transdermal, vaginal, transmucosal, or intestinal administration; parenteral delivery, including intramuscular, subcutaneous, intramedullary injections, as well as intrathecal, direct intraventricular, intravenous, intraperitoneal, intranasal, or intraocular injections, intracerebral among others. In some embodiments, methods for administration of a pharmaceutical composition comprising the PPARγ agonist include but are not limited to intracerebral direct injection, intracerebral pump infusion (e.g. by osmotic pump), intravenous, or intratumoral injection. Alternatively, the PPARγ agonist can be deposited at a site in need of treatment in any other manner.

In some embodiments, the method of administration encompasses features for steady-state regionalized delivery or accumulation at the site in need of treatment. In one example, the PPARγ agonist disclosed herein is delivered to a glioma by continuous intracerebral infusion using a mini-osmotic pump (e.g. an ALZETmini-osmotic pump (DURECT Corporation, Cupertino, Calif., U.S.A.)). These pumps can be filled with the PPARγ agonist in solution (e.g., DMSO), and will deliver the PPARγ agonist to the glioma in the brain by an osmotic displacement mechanism. Mini-osmotic pumps have a distinct advantage over direct injection for delivery of therapeutic agents such as PPARγ agonist because they maintain a well-defined and consistent pattern of delivery and tissue exposure over a significant period of time. Molecular weight, physical conformation, and chemical properties do not affect the delivery rate of a given compound.

In another aspect, the PPARγ agonist can also be administered to the glioma in the brain using a convection-enhanced drug delivery system, such as that described in U.S. Pat. No. 5,720,720, incorporated by reference herein. Convection-enhanced drug delivery involves positioning the tip of an infusion catheter within a tissue (e.g., brain tissue) and supplying the drug through the catheter while maintaining a positive pressure gradient from the tip of the catheter during infusion. The catheter is connected to a pump, which delivers the PPARγ agonist and maintains the desired pressure gradient throughout delivery of the drug. Drug delivery rates are typically about 0.5 to about 4.0 m/min with infusion distances of about 1 cm or more. This method is particularly useful for the delivery of drugs to the brain and other tissue, particularly solid nervous tissue. In certain embodiments, convection-enhanced drug delivery is useful for delivering PPARγ agonist in combination with a high molecular-weight polar molecule such as growth factors, enzymes, antibodies, protein conjugates and genetic vectors to the brain or other tissue. In these embodiments, inflow rates can be up to about 15.0 ml/min.

By way of example, an intracerebral cannula can be placed at an infusion site and anchored to the cranium, for example, with a cyanoacrylate adhesive using the ALZET brain infusion kit (Cupertino, Calif., USA) employing an Alzet 2004 osmotic pump. The osmotic pump can have a 2 ml volume and 0.25 μl/hour flow rate and can deliver the PPARγ agonist for a period of up to 4 wks. The pumps will be filled with drug or vehicle (DMSO, 0.1% final concentration in PBS). The pump can be surgically implanted subcutaneously in the subjects and secured by sutures. Doses of the PPARγ agonist can be delivered at a therapeutically effective dosage to the gliomas. (e.g., pioglitazone dosages of about 1 μM to about 30 μM).

For injections, the agents of the present invention may be formulated in solutions, such as dimethyl sulfoxide, and/or in physiologically compatible buffers such as Hank's solution, Ringer's solution, or physiological saline buffer. For such transmucosal administration, penetrants appropriate to the barrier to be permeated are used in the formulation. Such penetrants are generally known in the art.

In powders, the carrier is a finely divided solid which is in a mixture with the finely dived active component. In tablets, the active component is mixed with the carrier having the necessary binding properties in suitable proportions, which has been shaped into the size and shape desired.

The powders and tablets can contain from five or ten to about seventy percent of the active compounds. Carriers can include, but are not limited to, magnesium carbonate, magnesium stearate, talc, sugar, lactose, pectin, dextrin, starch, gelatin, tragacanth, methylcellulose, sodium carboxymethylcellulose, a low melting wax, cocoa butter and the like, among other embodiments (e.g., solid, gel, and liquid forms). The term “preparation” is intended to also encompass the formation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The molten homogenous mixture is then poured into convenient sized molds, allowed to cool, and thereby to solidify in a form suitable for administration.

Liquid form preparations include, but are not limited to, solutions, suspensions, and emulsions (e.g., water or water propylene glycol solutions). For parenteral injection, in some embodiments of the present invention, liquid preparations are formulated in solution in aqueous polyethylene glycol solution. Aqueous solutions suitable for oral use can be prepared by dissolving the active component in water and adding suitable colorants, flavors, and stabilizing and thickening agents, as desired.

Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form, the preparation is subdivided into unit doses containing appropriate quantities of the active component. The unit dosage form can be a packaged preparation, the package containing discrete quantities of preparation, such as paclceted tablets, capsules, and powders in vials or ampoules. Also, the unit dosage form can be a capsule, tablet, cachet, or lozenge itself, or it can be the appropriate number of any of these in packaged form.

The dose, amount, and/or quantity of PPARγ agonist or derivative thereof administered to the subject can depend on the specific PPARγ agonist or derivative thereof selected as well as the mode of administration. By way of example, for oral treatment a PPARγ agonist, such as pioglitazone, can administered to the subject at an approximate dosage of about 1 mg/kg/day to about 40 mg/kg/day, respectively.

The following examples are provided in order to demonstrate and further illustrate certain preferred embodiments and aspects of the present invention and are not to be construed as limiting the scope thereof.

In the experimental disclosure which follows, the following abbreviations apply: N (normal); M (molar); mM (millimolar); EM (micromolar); mol (moles); mmol (millimoles); μmolcromoles); nmol (nanomoles); pmol (picomoles); g (grams); mg (milligrams); μg (micrograms); ng (nanograms); l or L (liters); ml (milliliters); μl (microliters); cm (centimeters); mm (millimeters); μm (micrometers); nm (nanometers); ° C. (degrees Centigrade); Sigma (Sigma Chemical Co., St. Louis, Mo.), parts per million (ppm).

### EXAMPLE

### Inhibition of In Vivo Glioma Growth and Invasion by PPARgamma Agonist Treatment

In the present study, we demonstrate that the PPARγ agonist pioglitazone (pio) reduces cellular viability of C6 rat glioma and human glioma cell lines (A 172, U87) and inhibits C6 glioma cell proliferation, measured by Ki-67 expression, in vitro. Furthermore, pioglitazone treatment in PPARγ-cDNA overexpressing glioma cells reduced cellular viability in glioma cells, while treatment of glioma cells overexpressing the PPARγ mutant E499Q-cDNA, which lacks the transcriptional activity, shows no antineoplastic effects. These findings were confirmed in vivo using a C6 rat glioma model. Here tumor volumes were reduced by 83% following intracerebral pio administration and by 76.9% with oral pio treatment. In parallel, pio treated animals exhibited improved clinical outcome, a lower proliferation-index (Ki-67) and decreased Brd U-incorporation within the tumor tissue. In addition, in drug-treated animals tumors exhibited an upregulation of the proapoptotic proteins Bax and cleaved caspase-3 associated with increased TUNEL-labeling indicative of apoptotic cell death. Furthermore, reduced invasion, measured in vitro with Boyden chamber experiments and in vivo through MMP9 levels, was observed. Pio also induced upregulation of the astrocytic redifferentiation marker CS-56 in tumor cells in vitro and vivo as sign of induced redifferentiation.

### Material and Methods

### Material:

Pioglitazone (Takeda Chemical Industries, Osaka, Japan) was dissolved in dimethyl sulfoxide (DMSO) obtained from Sigma (St. Louis, Mo., USA). Dulbecco's modified Eagle's medium (DMEM), RPMI-1640 medium, penicillin, streptomycin, fetal calf serum, phosphate-buffered saline (PBS), trypsin-EDTA, and Proteinase K were purchased from Gibco (Gibco BRL, Karlsruhe, Germany). Rabbit Ki-67-antibody was purchased from NeoMarkers (Fremont, Calif., USA), rabbit cleaved caspase-3- and Rabbit BAX-antibody from Cell Signaling (Beverly, Mass., USA) goat anti-MMP9 from Santa Cruz (Santa Cruz, Calif., USA) and mouse anti-CS-56 from Sigma (St. Louis, Mo., USA). For western blot analysis the secondary anti-rabbit-antibody was obtained from Amersham Bioscience (Piscataway, N.J., USA). Secondary antibody for immunohistochemistry (Alexa Fluor© 488-conjugated goat anti-rabbit IgG) was purchased from Molecular Probes (Eugene, Oreg., USA).

### Cell Culture:

Rat C6 glioma cells were grown in DMEM and human glioma cells (U87, A172) in RPMI, supplemented with 10% (v/v) fetal calf serum, 100 U/mL penicillin and 100 U/mL streptomycin in a 5% CO₂ atmosphere. Primary astrocyte cultures were prepared as described previously (J Neurosci 1998; 18: 4451-60) and grown in DMEM, supplemented with 2.5% (v/v) fetal calf serum, 100 U/mL penicillin and 100 U/mL streptomycin in a 5% CO₂ atmosphere.

### Viability Assay:

Cellular viability was assessed by 3-[4,5-dimethylthiazol-2-yl]-2,5-diphenyltetrazolium bromide (MTT; Sigma, St. Louis, Mo., USA) assay. Briefly, C6 cells, U87 cells and A172 cells (5×10³/well) or primary astrocytes (5×10³/well) were seeded in a 96-well plate and exposed to different concentrations of pio (1, 10, and 30 μM; n=10). DMSO served as vehicle control (0.1% of final concentration). At 1, 3, 5, and 7 days, 10 μl MTT (5 mg/ml PBS) was added to each well and plates were incubated at 37° C. for 2 h. Medium was then removed and cells were resuspended in 100 μl DMSO. Cell viability was assessed by colorimetric change using Spectramax 340PC plate reader (Molecular Devices, Sunnyvale, Calif., USA) at λ=550 nm. Experiments were performed in triplicate.

### Transfection:

Transfections were carried out on human A172 glioma cells using Lipofectamine 2000 (Invitrogen) according to the manufactures instructions. PPARγ cDNA constructs were kindly provided by Dr. Ron Evans, The Salk Institute for Biological Studies, San Diego, USA (Mouse PPARgamma1 cDNA), from Dr. Evan Rosen, Harvard, USA (mouse PPARgamma2) from Dr. Bruce Spiegelman, Harvard, USA (mouse PPARgamma2 cDNA mutated at position E499Q31, a point mutation from Glu⁴⁹⁹ to Gln⁴⁹⁹ in the AF-2 activation domain, lacking its transcriptional activity). 48 h after transfection cells were treated with 30 μM pioglitazone. Cell viability was assessed after 5 d using the above-described MTT assay.

### Animals:

Spraque-Dawley rats (Charles River Laboratories, Sulzfeld, Germany), weighting 200-250 g were used. Animals were housed in groups of two under standard conditions at a temperature of 22° C.±1 and a 12-h light-dark cycle with free access to food and water. Experiments were carried out in accordance with the declaration of Helsinki and were approved by the local ethical committee for animal experiments.

### Brain Tumor Xenograft:

Before implantation, 85-90% confluent C6 cells were trypsinized, rinsed with DMEM+10% fetal calf serum, and centrifuged at 1000 rpm for 4 min. The cell pellet was resuspended in DMEM and placed on ice. Concentration of viable cells was adjusted to 1×10⁵ cells/1 μl of DMEM. Each rat was anesthetized and placed in a stereotactic frame (David Kopf Instruments, Tujunga, Calif., USA), a hole was drilled at AP 0.0, R −3.0 relative to bregma according to the stereotaxic atlas of König and Klippel (König and Klippel, 1963). Tumor cells were injected at a rate of 0.5 μl/s, using a 2 μl Hamilton (#2701) syringe (Reno, Nev., USA) with a 26 s-gauge needle mounted on a stereotactic holder at a depth of 5 mm. The needle was left in place for a further 5 min to prevent reflux along the needle tract. For intracerebral administration of pio/vehicle (DMSO), the tip of an Alzet-brain-infusion-kit (Cupertino, Calif., USA) was placed in a depth of 5 mm, connected to a 2ML4 Alzet osmotic pump (Cupertino, Calif., USA). Drug administration began 4 h after surgery. The osmotic pumps had a 2 ml volume and 2.5 μl/hour flow rate. The pumps were filled with 20 μM pio solved in PBS or vehicle (DMSO, 0.1% final concentration) solved in PBS. Animals were randomly distributed in two groups, treated with pio filled pumps (n=26) or vehicle filled pumps (n=26). Thereafter skull was cleaned and the incision sutured. For oral treatment the drug was pulverized, and mixed with Purina chow to give concentrations of 120 μm pioglitazone. Control animals received Purina chow without additions. Rats were allowed free access to the chow. Tumors were allowed to grow and animals were weighed daily. The intracerebral treated animals were sacrificed after 3 d (n=5/group), 6 d (n5/group), 9 d (n=5/group), 14 d (n=5/group), and 21 d (n=6/group), the orally treated animals after 21 d (n=8/group).

### Clinical Assessment:

After striatal injection of C6 cells and intracerebral treatment with pio or vehicle for 21 d, the animals were examined for neurological deficits. Neurological function was quantified by evaluation of hemiparesis, cycling and immobility (J Neurosurg 1986; 65: 222-9, Stroke 1999; 30: 427-31; discussion 431-2). Hemiparesis was assessed by forelimb flexion and immobility by the loss of ability to walk a distance greater than 15 cm. One point was given for each negative finding and total score then computed. The scores of the treated and untreated group were averaged. A blinded observer performed neurological examinations.

### Sections:

Brains were serially sectioned at 10 μm using a cryostat (Leica, Jung CMI800, Germany). Sections for hematoxylin-eosin (HE)-staining were placed onto uncoated slides. Sections intended for use in immunoreactivity assays were placed onto coded slides (Fisher finest, premium; Houston, Tex., USA). Sections were routinely HE-stained for histomorphological assessment and measurement of tumor volume as well as immunohistochemically processed for Ki-67-, BAX, cleaved caspase-3-expression, MMP9, CS-56 and BrdU-incorporation.

### Tumor Volume:

Images of HE-stained sections containing the tumor were captured with a SPOT model 1.3 camera (Diagnostic Instruments, Inc., Sterling Heights, Mich., USA) using a 1× objective and images processed using NIH Image 1.62 software (Bethesda, Md., USA). The tumor area of each section was manually outlined by a blinded observer using the freehand selection tool to measure tumor area in mm². The area was then multiplied by the section thickness (10 μm section, 10 sections/HE stain) to achieve a section volume measurement. Volumes of all sections were added to calculate the total volume of each tumor. Tumor volumes for five/six animals of every group were measured.

### Western-Blot:

For western-blot analysis, tumor-containing hemispheres were homogenized in Tris-HCl [50 mM Tris-HCl pH8, 120 mM NaCl, 5 mM EDTA, 0.5% (v/v) NP-40, 160 mM phenylmethylsulfonyl fluoride (PMSF)] and sonicated. Homogenates were collected by centrifugation (15 min 11000 g. 4° C.) and protein concentration in the supernatant was determined using the Bio-Rad Protein Assay (Biorad, Munich, Germany). Lysates (20-40 μg) were separated on a 7% (w/v) sodium dodecyl sulfate (SDS)-polyacrylamide gel under reducing conditions and transferred to a PVDF membrane (Millipore, Bedford, Mass., USA). Non-specific binding was blocked by incubation with 5% (w/v) skimmed milk in TBS for 2 h. Following incubation with the primary antibody overnight at 4° C. [rabbit anti-BAX 1:1000, rabbit anti-cleaved caspase-3 1:1000, mouse anti-CS-65 1:1000, goat anti-MMP-9 1:1000 in TBS containing 0.1% (v/v) Tween 20] membranes were washed three times in TBS/Tween for 5 min and subsequently incubated for 120 min in TBS/Tween containing secondary peroxidase-conjugated antibody at room temperature (anti-rabbit 1:1000, anti-mouse 1:1000; anti-goat 1:1000 respectively). Signals were visualized by chemoluminescence (Pierce, Rockford, Ill., USA) and band intensities were quantified with NIH Image 1.62 software (Bethesda, Md., USA).

### Immunohistochemistry:

Frozen brain sections were first blocked with 5% normal goat serum or normal horse serum in PBS and subsequently incubated with rabbit anti-Ki-67 antibody (1:200 dilution), mouse anti-CS-56 (1:100 dilution), goat anti-MMP-9 (1:100 dilution) or rabbit anti-cleaved caspase-3 antibody (1:200 dilution) at 4° C. overnight. Sections were washed extensively with PBS before incubation with secondary antibodies. Incubation was carried at room temperature for 1 h. After washing with PBS three times, stained slides were mounted with PBS/glycerol (1:1) and viewed under a fluorescent microscope (Leica, Germany).

C6 glioma cell cultures were treated with pio or vehicle for 2 d and 5 d and fixed in 4% paraformaldehyde, rinsed in TBS and incubated with rabbit anti-Ki-67 (1:200 dilution) at 4° C. overnight and processed as described above. Counterstaining was carried out with propidium iodide (P1; 1:40; Sigma, St. Louis, Mo., USA) or DAPI-staining (1:500 in PBS).

For determination of the proliferation-index, Ki-67 positive cells in pio and vehicle treated cells and animals were counted, the percentage of Ki-67 positive cells in 1×10³ tumor cells (P1 staining used in the in vitro experiments—not shown; DAPI stain used in the in vivo experiments) calculated and statistically compared. The percentage of cleaved caspase-3 positive cells was assessed by counting the immunopositive cells in 1×10³ tumor (DAPI stain) cells and statistically compared.

Terminal Deoxynucleotidyl Transferase-Mediated dUTP Nick-End Labeling Assay for Apoptosis:

Frozen brain tissue sections were examined for apoptosis using the terminal deoxynucleotidyl transferase-mediated dUTP transferase nick-end labeling (TUNEL assay). Apoptosis was evaluated using the Promega DeadEnd Fluorometric Tunel System (Promega, Madison, Wis., USA) according to the manufacturer's instructions. The percentage of TUNEL positive cells was assessed by counting the immunopositve cells in 1×10³ tumor (DAPI stain) cells and statistically compared.

### BrdU Incorporation:

At thirteen days of treatment animals were intraperitoneally injected with 300 mg/kg of 5-bromodeoxyuridine (BrdU; Sigma, St. Louis, Mo.), a thymidine analog that incorporates into the DNA of dividing cells during S phase and can be detected immunohistochemically. Twenty-four hours later, these animals were anesthetized and perfused with phosphate-buffered saline (PBS). Brains were dissected, frozen, cut and immunostained in the following manner: Sections were fixed in 4% paraformaldehyde, washed in PBS, and incubated in 2 N HCl for 10 min at room temperature. Sections were again washed in PBS and incubated in BrdU primary antibody (rat monoclonal 1:100, Abcam, Cambridge, UK) in PBS containing 5% normal goat serum overnight at 4 C. After washing with PBS, sections were incubated in rhodamine-labeled anti-rat secondary antibody (1:100, Jackson, West Grove, Pa., USA) in PBS containing 5% normal goat serum for 1 h at room temperature. The number of dividing cells present in the tumor of these BrdU-stained sections was counted and compared to total amount of tumor cells stained with DAPI.

### Boyden-Chamber-Assay:

C6 cell migration in the presence or absence of pio was assessed using a Boyden chamber (AP48, NeuroProbe, Gaithersburg, Md., USA) with an 8 μm polycarbonate PVPF-filter (Osmonics, Minnetonka, Minn., USA). Cells were suspended in DMEM containing 2.5% fetal calf serum and pio at 30 μM or vehicle (DMSO). C6 glioma cells were pretreated for 24 h in the presence or absence of 30 μM pio. Cells (5×10³ in 50 μl pio/medium or vehicle/medium) were then plated in the wells of the upper compartment of the chamber (6 well/condition), and the wells of the lower compartment were filled with DMEM. Incubation was performed at 37° C. in 5% CO₂ for 4 h. After incubation, cells on the upper surface of the filter, which had not migrated, were gently scraped off, and the filters were then fixed in methanol and subsequently stained with DAPI (1:500 in PBS; Sigma, St. Louis, Mo., USA). The number of cells which migrated to the lower surface of the filter, was counted using public domain software NIH Image 1.62 (Bethesda, Md., USA). Total number of migrating cells under pio treated and vehicle treated condition were compared statistically. Experiments were performed in duplicate.

### Statistical Evaluation:

Cellular viability data, tumor volumes, immuno-positive cells, clinical scores, proliferation-index and densitometric results of Bax and cleaved caspase-3 western-blots were analyzed by student-T-test using Prism version 3.00 software (GraphPad Software, San Diego, Calif., USA).

### Results:

### Reduced Cellular Viability of Rat C6 Glioma and Human Glioma Cells:

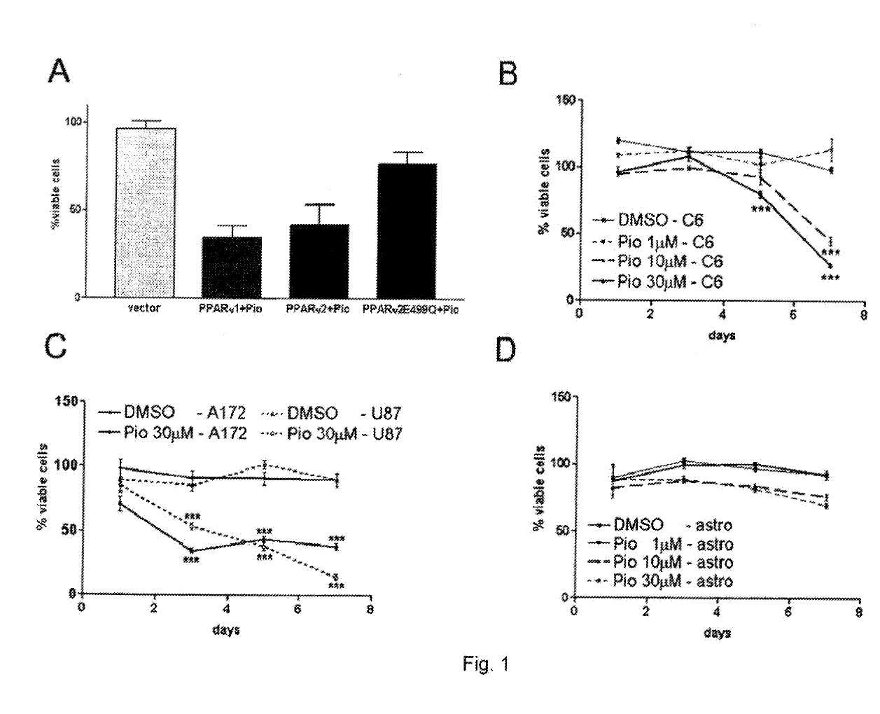

Viability of rat C6 glioma cells and human glioma cells (U87, A172) was assessed after incubation with increasing concentrations (C6: 1, 10, 30 μM; U87/A172: 30 μM) of the PPARγ agonist pio at day 1, 3, 5, and 7 (FIGS. 1A, B). Ten and 30 μM pio significantly reduced the cellular viability of C6 rat glioma cells in a concentration- and time-dependent manner (FIG. 1A). A significant reduction of cellular viability in C6 cells was observed at 5 d and 7 d after treatment with 30 μM pio and at 7 d after treatment with 10 μM pio (FIG. 1A). At 7 d, 30 μM of pio reduced viability to 31%±3.7, and 10 μM of pio to 52.1%±6.4 (FIG. 1A). In the human glioma cell lines U87 and A1 72, 30 μM pio significantly reduced cellular viability at 3, 5, and 7 d (FIG. 1B).

At all time points and concentration evaluated, viability of primary astrocytes was not affected by pio treatment (FIG. 1C), indicating that PPARγ ligand-mediated cell death is restricted to neoplastic astrocytic cells.

To assess the role of PPARγ in the antineoplastic effects of pio, we overexpressed PPARγ in the human glioma cell line A172. Overexpression of two different PPARγ-cDNA, PPAR1 and 2, reduced cellular viability of A172 cells measured by MTT compared to medium and vector control. After transfection of A172 cells with a PPARγ mutant-cDNA lacking transcriptional activity the decreasing effects on cellular viability where no longer observed at 5 d following transfection (FIG. 1D).

Ki-67 Immunoreactivity and Proliferation-Index is Reduced after Pioghtazone Treatment In Vitro:

To further characterize the pio-induced effects, Ki-67 expression, a marker for tumor proliferation and malignancy, was evaluated. C6 cells were treated with 30 μM pio or vehicle and Ki-67 immunoreactivity was assessed at 2 d (supplement FIGS. 1A, B) and 5 d (supplement FIGS. 1C, D). Pio reduced the number of Ki-67 immunopositive cells compared to vehicle treatment at both time points. Thus, the fraction of Ki-67 positive, proliferating cells, expressed as proliferation-index, was significantly reduced after 5 d of pio treatment (supplement FIG. 1E).

### Significantly Reduced Tumor Volume and Growth in the Rat Glioma Model:

In order to confirm the antineoplastic effects of pio in vivo, C6 glioma cells were injected in rat striata and treated with pio 20 μM or vehicle via an osmotic pump for 3 d (n=5/group), 6 d (n=5/group), 9 d (n=5/group), 14 d (n5/group), or 21 d (n=6/group) starting 4 h after initial tumor cell injection.

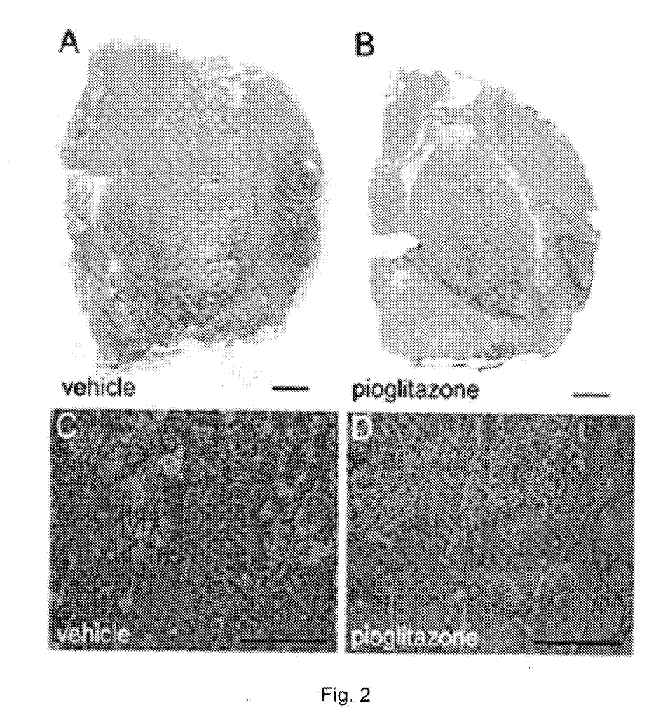

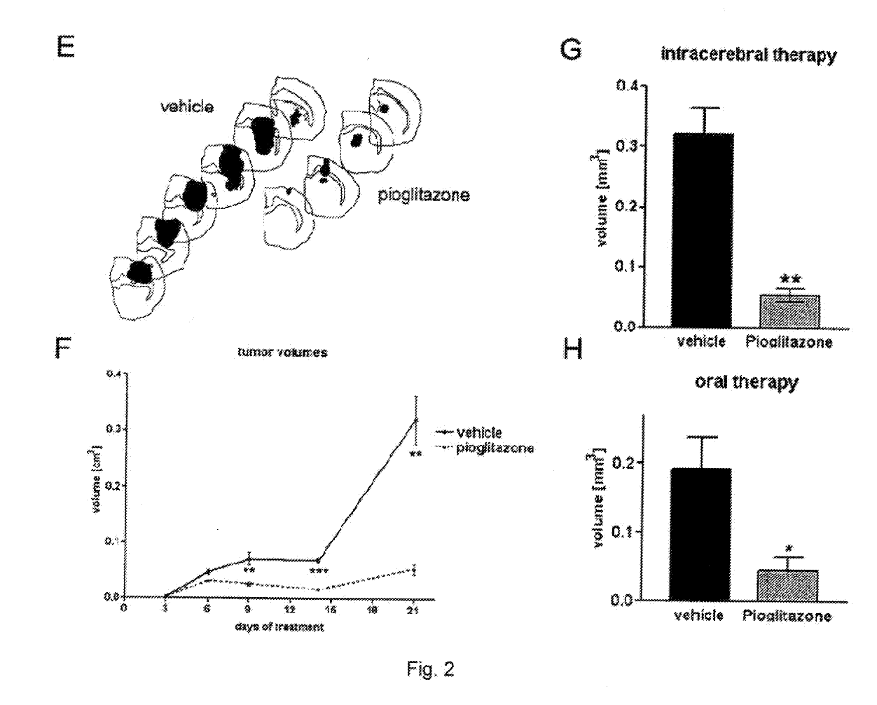

The pio-treated animals showed a dramatic reduction of tumor volume at 21 days of treatment (FIGS. 2B, D, E) compared to vehicle-treated animals (FIGS. 2A, C, E). Vehicle-treated animals exhibit a median tumor volume of 0.321 cm³±0.11 whereas pio-treated animals revealed tumor volumes of 0.055 cm³±0.03, reflecting an 83% reduction (FIG. 2G). At 3 and 6 days no significant differences in tumor volumes could be observed (FIG. 2F). However, at 9, 14, and 21 days tumor volumes decreased significantly in pio treated animals (FIG. 2F). These effects could also be observed in the oral treated animals, where pio treatment reduced tumor volumes by 76.9% after 21 d (FIG. 2H).

### Improved Clinical Outcome:

To investigate whether the reduction of tumor volumes results in an improved neurological outcome, animals were assessed and scored for signs of hemiparesis, cycling and immobility as previously described by (J Neurosurg 1986; 65: 222-9; Stroke 1999; 30: 427-31; discussion 431-2). Pio-treated animals had a significant better clinical outcome overall. They exhibited less hemiparesis, no cycling, and less immobility (supplement FIG. 2) then vehicle treated control animals.

Animals were also closely monitored for weight-loss, a non-behavioral indicator of intracellular tumor growth (Lab Anim Sci 1991; 41: 269-73). Daily weighing of animals showed the vehicle-treated group exhibiting a significant weight loss at 17 d, whereas pio-treated animals did not show any weight loss (supplement FIG. 2E). Together, these clinical evaluations suggest that pio-induced reduction of tumor volume improved clinical outcome.

### Induction of Apoptotic Cell Death In Vivo:

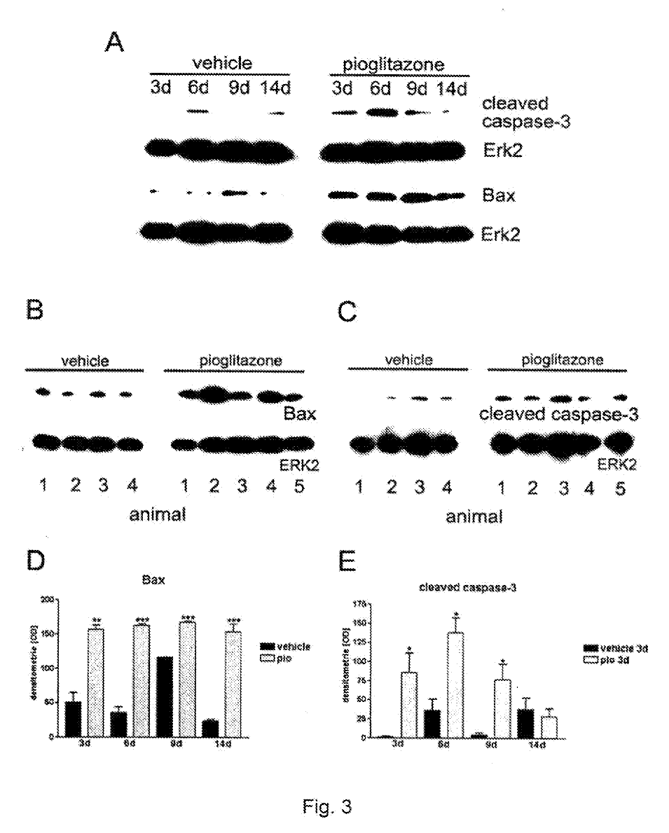

To assess if pio induces apoptotic cell death in vivo Bax- and cleaved caspase-3-expression were evaluated and TUNEL staining was performed. The expression of the proapoptotic proteins Bax and cleaved caspase-3 was detected in whole-hemisphere lysates of C6 glioma animals and controls. Analysis at different time points revealed that both proteins were expressed in the pio treated animals and in controls (FIG. 3A). Protein levels of either Bax or cleaved caspase-3 were upregulated in the early phase of pio treatment. Densitometric analysis showed a significant difference in Bax-protein levels at 3, 6, 9, and 14 d (FIG. 3D) and in cleaved caspase-3-protein levels at 3, 6, and 9 d (FIG. 3E) in response to pio treatment. At 6 d of pio treatment Bax and cleaved caspase-3 were upregulated (FIG. 3B) with a 1.66 fold induction of Bax protein (FIG. 3D) and a 2.88 fold induction of cleaved caspase-3 (FIG. 3E).

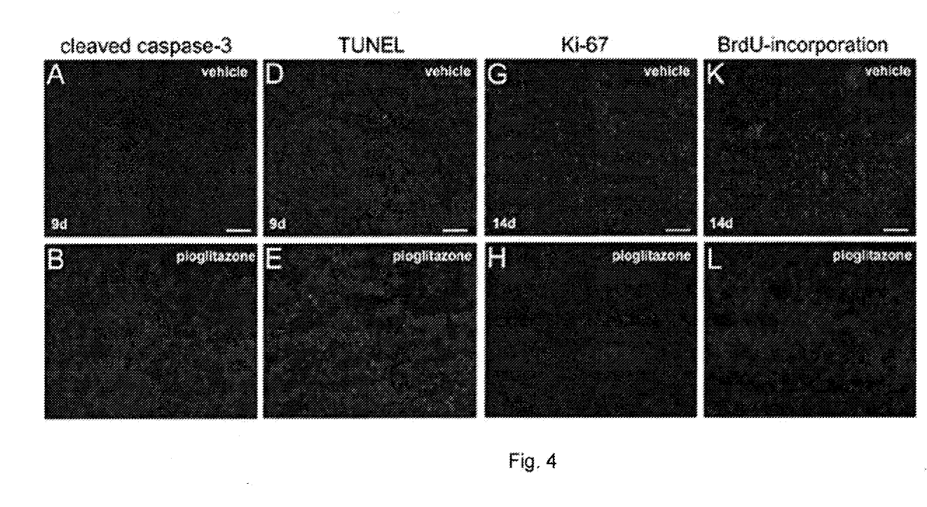

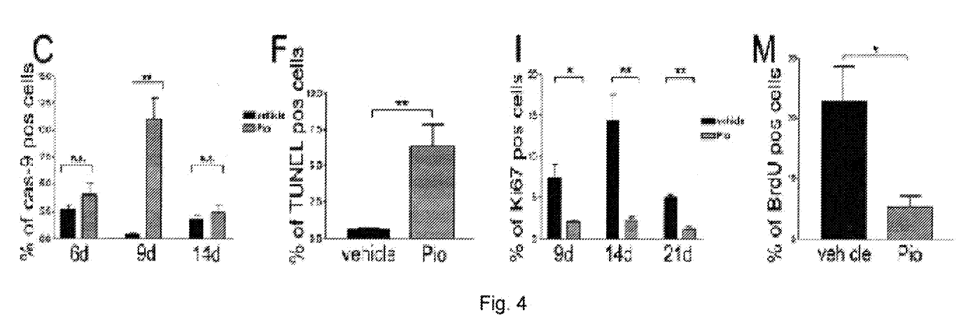

Immunohistochemistry revealed elevated cleaved caspase-3 expression at 6 to 14 days of pio treatment with a significant maximum at 9 d (FIG. 4) compared to vehicle treated animals (FIG. 4). DAPI staining was used to tag single tumor cells and to determine the percentage of cleaved caspase-3 positive cells (FIG. 4). Increased TUNEL staining was detected at 9 days of pio treatment (FIG. 4). Very few cells were positively TUNEL stained in vehicle controls (FIG. 4).

### Ki-67 Immunoreactivity and Proliferation-Index is Reduced In Vivo:

Ki-67 expression was used to evaluate degree of tumor proliferation in vivo. C6 glioma animals showed significant reduction of Ki-67 expression at 9, 14, and 21 days of pio treatment (FIG. 4) compared to vehicle-treated animals (FIG. 4). The proliferation-index, calculated from the percentage of Ki-67 positive cells in 1×10³ tumor cells (DAPI), was reduced by pio treatment at 9, 14 and 21 days (FIG. 4) confirming the results obtained in vitro.

### Reduction of BrdU-Incorporation:

To further confirm that pio treatment reduces proliferation, we assessed BrdU-incorporation in vivo. BrdU-incorporation was reduced in response to pio treatment at 14 d (FIG. 4) compared to the vehicle treated groups (FIG. 4). The percentage of BrdU positive cells is significantly lower in pio treated animals (FIG. 4).

Invasion of C6 Rat Glioma Cells after Pioglitazone Treatment In Vitro:

In addition to proliferation, the ability of tumor cells to invade healthy near-by tissue is characteristic of the malignancy state of gliomas. Histological evaluation of pio treated in vivo gliomas revealed more defined tumor margins and decreased invasiveness (FIGS. 2C,D). This led us to examine invasiveness of C6 glioma cells using a Boyden chamber assay. Pio-pretreated (30 μM) (supplement FIGS. 3D, E, F) and untreated (supplement FIGS. 3A, B, C) C6 glioma cells were suspended in the upper chamber containing pio at 30 μM (supplement FIGS. 3B, E) or vehicle (supplement FIGS. 3A, D). At 4 h, cells that migrated to the other side of an 8 μm filter were stained with DAPI and counted. Pio treatment during the Boyden chamber incubation reduced the invasiveness of C6 cells significantly (supplement FIGS. 3A, B, C). Pretreatment of C6 cells for 24 h with pio further increased inhibition (supplement FIGS. 3D, E, F).

### MMP9 and CS-56 Expression In Vivo:

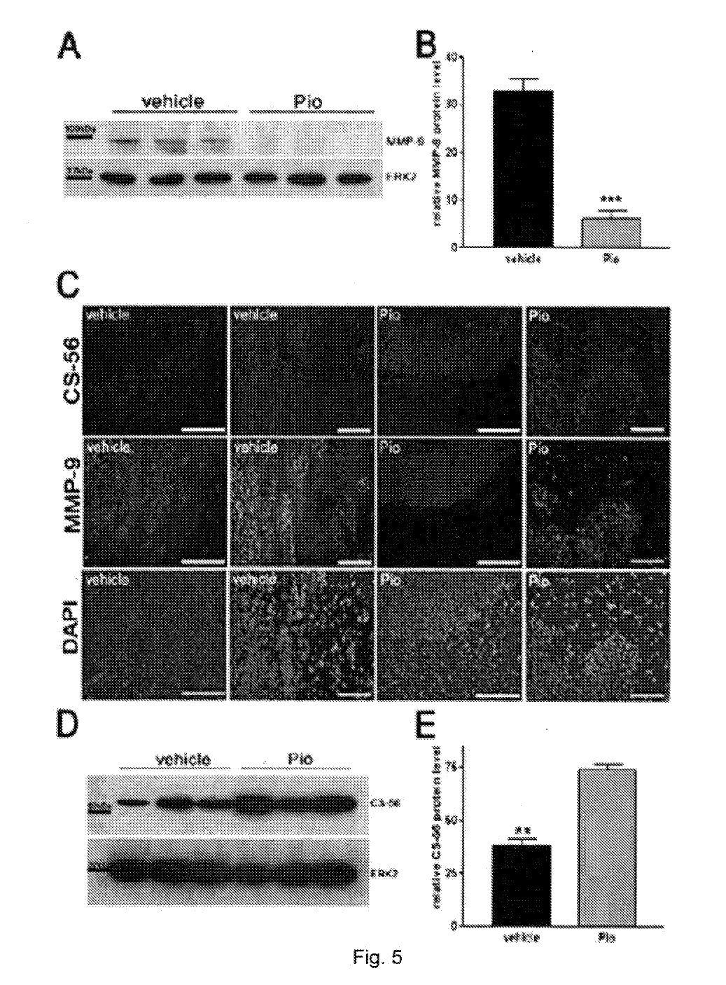

In order to characterize the invasion of pio-treated tumors in vivo, we investigated levels of MMP-9, a protein which plays a major role in glioma invasion (Cancer Res 1993; 53: 2208-11). MMP-9 expression was detected in whole-hemisphere lysates of C6 glioma animals using a goat-antiMMP-9-antibody. MMP-9 expression was almost completely suppressed in pio-treated animals (FIG. 5A). Erk2 expression served as a loading control (FIG. 5A). Densitometric analysis showed an 81% reduction of MMP-9 expression in the pio-treated group (FIG. 5B). Immunoreactivity of MMP-9 revealed that the epitope is only expressed in the tumor and adjacent brain regions (FIG. 5C). Pio treatment reduced MMP-9 positive staining at tumor margins and even more so in the surrounding healthy brain tissue compared to vehicle treatment (FIG. 5C).

The expression of a marker of astrocytic differentiation, CS-56 (Cell Tissue Res 2001; 306: 15-26), was evaluated in the tumors (FIG. 5C). An upregulation of CS-56 was most prominently observed at the tumor margin towards healthy brain tissue compared to vehicle-treated animals (FIG. 5C). Parallel analysis of tissue lysates by western-blot (FIG. 5D) revealed upregulation of CS-56 expression in pio-treated animals, which was corroborated by the densitometrical measurement, showing an increase of around 56% in CS-56 protein levels (FIG. 5E). Erk2 expression served as a loading control (FIG. 5D).

### Discussion

The antineoplastic effects of different PPARγ agonists on human tumor cells (Review) and rat glioma cell lines (J Pharmacol Exp Ther 2005; J Neuromchem 2002; 81: 1052-60) led us to question if the synthetic PPARγ agonist pioglitazone (pio) could exert similar effects in rat glioma and human glioma cells as well as in an appropriate glioma model. Pio is a member of the thiazolidinedione (TZD) class of new antidiabetic drugs and was chosen since it is already in clinical use for treatment of type II diabetes mellitus (ACTOS®) and would be readily available for glioma therapy in clinical studies. In the present study pio demonstrated antineoplastic potency in vitro by decreasing cellular viability of both human and rat glioma cells. Similar effects were found in PPARγ overexpressing glioma cells, whereas pio treatment of glioma cells overexpressing a PPARγ-mutant, lacking the transcriptional activation, did not induce antineoplastic changes. We demonstrated that the reduction of cellular viability by pio is restricted to neoplastic cell types, as primary astrocytes were not affected by pio treatment.

Of three glioma cell lines we studied, PPARγ protein levels are highest in U87 compared to A176 and C6 (J Neurochem 20020; 81: 1052-60). Pio reduced cellular viability of the human glioma cell line U87 more robustly than either A172 or C6 glioma cell lines, a phenomenon that may be related to the relative PPARγ protein levels expressed in these glioma cell lines. In comparison to A176 and C6 cells PPARγ protein levels are high in U87 cells.

To characterize pio-induced effects on gliomas, Ki-67 immunoreactivity in C6 cells following pio treatment was evaluated. Ki-67 is a nuclear protein expressed in proliferating cells and serves as an important neuropathological marker for diagnosis of human gliomas (Clin Neuropathol 2002; 21: 252-7). Cells in GO (quiescent) phase are negative for Ki-67 (Histopathology 2002; 40: 2-11) and its expression in the normal brain is very low. In astrocytomas, Ki-67 expression is upregulated and correlates well with tumor grade and clinical prognosis, thus serving as a useful measure of active tumor volume and margin (Histopathology 2002; 40: 2-11; Br J Neurosurg 1991; 5: 289-98). Pio treatment reduced Ki-67 expression and diminished the proliferation index in vitro. Therefore, pio reduces both cellular viability and proliferation, leading to a reduced tumor cell number in vitro.

To verify these in vitro results in vivo, C6 cells were injected into rat striata (Gene Ther 1998; 5: 1187-94) and either continuously treated with pio through an intracerebrally placed osmotic pump or orally for three weeks. Intracerebral pio treatment of the induced intrastriatal tumors reduced tumor volumes by 83%, oral pio treatment by 76.9% compared to vehicle-treated animals. Furthermore, preservation of neurological function and body weight was observed in the pio-treated animals. Monitoring of tumor volumes showed no significant difference between treated and untreated groups during the early treatment period (3 and 6 days) indicating that pio treatment did not affect initial tumor inoculation. At 9 days tumor volumes were increased in the vehicle group and volumes decreased in the pio treated animals. This may indicate that antineoplastic effects of pio appear between days 6 to 9 in vivo, a time frame identical to our results in vitro.

With glioma tumor growth elevation of intracerebral pressure and corresponding tissue damage normally impair neurological performance. Examination of the pio-treated animals revealed an overall improvement of clinical outcome. In particular, examination revealed less hemiparesis and immobility and no instance of cycling. Pio treated animals also resisted weight loss in the last days of treatment. Weight loss was pronounced in untreated animals and further underlines the poor clinical condition of the vehicle-treated animals (Lab Anim Sci 1991; 41: 269-73). Pio treatment not only reduced tumor volumes and tumor malignancy but also improved tumor-related aggravation of clinical symptoms.

We investigated Bax protein expression because initial in vitro data indicate that PPARγ-agonists mediate their antineoplastic effects in rat and human glioma cell lines through a Bax upregulation with an induction of apoptosis, which could be abolished by Bax antisense-oligonucleotides (J Neurochem 2002; 81: 1052-60). Because primary data revealed tumor reduction by pio was not associated with an upregulation of proapoptotic proteins after 21 days of treatment (data not shown) earlier time points were evaluated. Bax as well as its downstream target cleaved caspase-3 were upregulated by pio over time, peaking in the early phase of the treatment. At 6 days of pio treatment a significant induction of these proapoptotic proteins was observed, which was correlated with an increase in cleaved caspase-3 immunoreactivity, reaching a significant difference at day 9 of treatment. Similarly, cleaved caspase-3-protein expression peaked at 9 days of pio treatment and decreased thereafter. Correlating to this finding, a significant increase in TUNEL labeling could be found at 9 days of pio treatment. Together, these findings indicate, that pio induced apoptosis of rat C6 gliomas occurred from days 6 to 9 and contributed at least in part to the observed reduction in tumor volume.

Reduced tumor growth might also be due to decreased proliferation, as observed in vitro. To assess if pio also reduces proliferation in vivo Ki67-expression and BrdU-incorporation rate was determined since earlier studies showed the ability of PPARγ agonists to reduce BrdU-incorporation in various cell lines having defective cell cycle regulators (Oncogene 2003; 22: 4186-93). Both proliferation markers were reduced by pio treatment, indicating that pio not only induced apoptosis but also reduced proliferation. Therefore, it seems likely both mechanisms, proliferation reduction and apoptosis account for decreased tumor volumes in animals receiving pio.

Histological differences, better defined tumor margins and fewer invasive cells, following pio treatment led us to test pio-induced reduction of C6 cell invasion in vitro using the Boyden chamber assay. This assay reliably tests drug effects by monitoring C6 cell migration and invasion (Invasion Metastasis 1998; 18:142-54). Pio treatment resulted in dramatically reduced cell migration in the Boyden chamber assay. These results clearly demonstrate the ability of pio to reduce both cell migration and invasion.

Furthermore, matrix-metalloproteinases play a critical role in tumor invasiveness and in the malignancy of gliomas (Cancer Res 1993; 53: 2208-11) by mediating basal membrane breakdown (J Neurooncol 2001; 53: 213-35). MMP-9, the most abundant MMP in gliomas (Br J Cancer 1999; 79: 1828-35), is elevated during tumor progression (Cancer Res 1993; 53: 2208-11) due to its secretion by glioma cells (Clin Cancer Res 2002; 8: 2894-901). MMP-9 levels are also highly correlated with the histological grade of malignancy (Cancer Res 1993; 53: 2208-11). Additionally, both MMP-9 mRNA and protein levels are elevated in biopsies of glioma patients (Clin Cancer Res 2002; 8: 2894-901).

Using N-cadherin as an in vitro marker of astrocytic differentiation, Zander et at. (J Neurochem 2002; 81: 1052-60) showed that treatment of C6 cells with the PPARγ agonist ciglitazone lead to increased redifferentiation. Therefore, we investigated protein levels and immunohistochemical localization of CS-56, as astrocytic redifferentiation marker, in treated and untreated animals. CS-56 expression reflects levels of chondroitin sulfate proteoglycans, the most abundant component of the extracellular matrix of mammalian brain (Neuron 1990; 4: 949-61). Chrondroitin sulfate proteoglycans are involved in development, regulation of proliferation, and redifferentiation (Cell Tissue Res 2001; 306: 15-26). CS-56 is located predominantly at the tumor border, where it is highly upregulated by pio treatment both in vitro (data not shown) and in vivo. Pio may therefore not only induce cell death and inhibition of proliferation in neoplastic cells but may also elicit redifferentiation in the malignant cells. The CS-56 upregulation at the tumor margin could also reflect glia formation separating tumor tissue from healthy brain tissue because CS-56 is strongly upregulated in glial scars (Nature 1997; 390: 680-3).

The concentrations of pio required to affect cellular viability are higher than expected according to its known in vitro receptor binding affinity (Biochem Biophys Res Commun 2000; 278: 704-11). Although pio is a synthetic PPARγ agonist and produces its receptor dependent effects on adipocytes, PPARγ independent mechanisms have also been reported for thiazolidinediones (Nat Med 2001; 7: 48-52) and need to be considered. PPARγ agonists like troglitazone inhibit cholesterol biosynthesis (Diabetes 1999; 48: 254-60) or acyl-CoA synthetase in a PPARγ-independent way (J Biol Chem 2001; 276: 33736-40). Moreover, thiazolidinediones inhibit proliferation in PPARγ knock-out mouse embryonic stem cells (Cancer Res 2001; 61: 6213-8). PPARγ-independent thiazolidinedione effects require higher concentrations as expected by in vitro experiments. These data indicate that, although thiazolidinediones are PPARγ agonists and PPARγ-mediated differentiation through transcriptional activity is part of their antineoplastic effects, PPARγ levels and receptor activation may not exclusively mediate the observed antineoplastic effects. Therefore, the described effects of pio on gliomas could be due to a PPARγ-dependent or independent mechanism. PPARγ-cDNA overexpression alone is able to induce reduction of cellular viability in glioma cells, which can not be seen by overexpressing the PPARγ mutant E499Q, with a mutation in the transcriptional AF-2 activation domain, indicating that the described pio effects indeed are displayed in a PPARγ dependant way.

Drugs that reduce cell proliferation as well as invasion and induce tumor cell death and redifferentiation, can be used to treat human tumors. Although the molecular basis of antineoplastic mechanisms of PPARγ agonists are yet not fully understood, the thiazolidinediones offer a new therapeutic approach in human glioma therapy because of their negative effects on proliferation and invasion as well as their positive effects on apoptosis and redifferentiation.

From the above description of the invention, those skilled in the art will perceive improvements, changes and modifications. Such improvements, changes and modifications within the skill of the art are intended to be covered by the appended claims. All references, publications, and patents disclosed above are herein incorporated by reference in their entirety.

Having described the invention, the following is claimed:

1. A method of treating a central nervous system tumor in a subject, the method comprising: administering a therapeutically effective amount of at least one PPARγ agonist or a derivative thereof to the tumor in the subject.

2. The method of claim 1, the PPARγ agonist or derivative thereof being administered to the subject in an amount effective to induce upregulation of proapoptotic protein Bax and cleaved caspase-3 in the tumor.

3. The method of claim 1, the PPARγ agonist or derivative thereof being administered at an amount effective to suppress expression of MMP-9 in the tumor of the subject.

4. The method of claim 1, the PPARγ agonist or derivative thereof being administered at an amount effective to mitigate tumor cell proliferation.

5. The method of claim 1, the PPARγ agonist or derivative thereof being administered directly to tumor of the subject by intracerebral infusion.

6. The method of claim 1, the PPARγ agonist or a derivative thereof comprising a compound of Formula I or pharmaceutically acceptable salt of a compound of Formula I, wherein Formula I is: 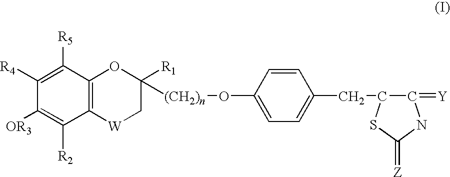
wherein R₁ and R₂ are the same or different, and each represents a hydrogen atom or a C₁-C₅ alkyl group;
R₃ represents a hydrogen atom, a C₁-C₆ aliphatic acyl group, an alicyclic acyl group, an aromatic acyl group, a heterocyclic acyl group, an araliphatic acyl group, a (C₁-C₆ alkoxy)carbonyl group, or an aralkyloxycarbonyl group;
R₄ and R₅ are the same or different, and each represents a hydrogen atom, a C₁-C₅ alkyl group or a C₁-C₅ alkoxy group, or R₄ and R₅ together represent a C₁-C₅ alkylenedioxy group;
n is 1, 2, or 3;
W represents the CH₂, CO, or CHOR₆ group in which R₆ represents any one of the atoms or groups defined for R₃; and
Y and Z are the same or different and each represents an oxygen atom or an imino (—NH) group; and pharmaceutically acceptable salts thereof.

7. The method of claim 1, the PPARγ agonist or a derivative thereof comprising a compound of Formula II or pharmaceutically acceptable salt of a compound of Formula II, wherein Formula II is: 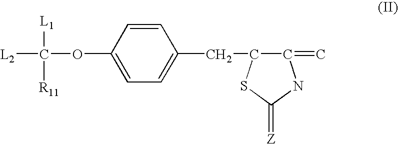
wherein R₁₁, is a substituted or unsubstituted alkyl, alkoxy, cycloalkyl, phenylalkyl, phenyl, aromatic acyl group, a 5- or 6 membered heterocyclic group including 1 or 2 heteroatoms selected from the group consisting of nitrogen, oxygen, and sulfur, or a group of the formula indicated in: 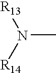
wherein R₁₃ and R₁₄ are the same or different and each is lower alkyl; and
wherein L¹ and L² are the same or different and each is hydrogen or lower alkyl or L¹ and L² are combined to form an alkylene group.

8. The method of claim 1, the PPARγ agonist or a derivative thereof comprising a compound of Formula III or pharmaceutically acceptable salt of a compound of Formula III, wherein Formula III is: 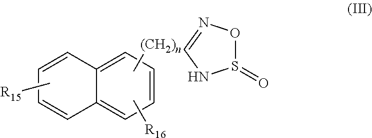
wherein R₁₅ and R₁₆ are independently hydrogen, lower alkyl containing 1 to 6 carbon atoms, alkoxy containing 1 to 6 carbon atoms, halogen, ethyl, nitrite, methylthio, trifluoromethyl, vinyl, nitro, or halogen substituted benzyloxy; and n is 0 to 4.

9. The method of claim 1, the PPARγ agonist or a derivative thereof comprising a compound of Formula IV or pharmaceutically acceptable salt of a compound of Formula IV, wherein Formula IV is: 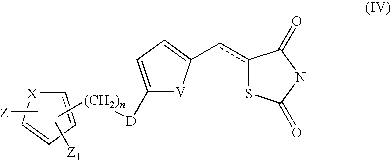
wherein the dotted line represents a bond or no bond; V is HCH—, —NCH—, —CH═N—, or S;
D is CH₂, CHOH, CO, C═NOR₁₇, or CH═CH; X is S, SO, NR₁₈, —CH═N, or —N═CH;
Y is CH or N;
Z is hydrogen, (C₁-C₇)alkyl, (C₁-C₇)cycloalkyl, phenyl, naphthyl, pyridyl, furyl, thienyl, or phenyl mono- or di-substituted with the same or different groups which are (C₁-C₃)alkyl, trifluoromethyl, (C₁-C₃)alkoxy, fluoro, chloro, or bromo
Z₁ is hydrogen or (C₁-C₃)alkyl;
R₁₇ and R₁₈ are each independently hydrogen or methyl; and n is 1, 2, or 3.

10. The method of claim 1, the PPARγ agonist or a derivative thereof comprising a compound of Formula V or pharmaceutically acceptable salt of a compound of Formula V, wherein Formula V is: 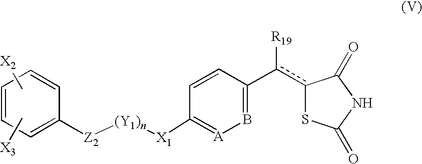
wherein the dotted line represents a bond or no bond;
A and B are each independently CH or N with the proviso that when A or B is N the other is CH; X is S, SO, SO₂, CH₂, CHOH, or CO;
n is 0 or 1;
Y₁ is CHR₂₀ or R₂₁, with the proviso that when n is 1 and Y₁ is NR₂₁, X₁ is SO₂ or CO; Z₂ is CHR₂₂, CH₂CH₂, cyclic C₂H₂O, CH═CH, OCH₂, SCH₂, SOCH₂, or SO₂CH₂;
R₁₉, R₂₀, R₂₁, and R₂₂ are each independently hydrogen or methyl; and
X₂ and X₃ are each independently hydrogen, methyl, trifluoromethyl, phenyl, benzyl, hydroxy, methoxy, phenoxy, benzyloxy, bromo, chloro, or fluoro.

11. The method of claim 1, the PPARγ agonist or a derivative thereof comprising a compound of Formula II or pharmaceutically acceptable salt of a compound of Formula VI, wherein Formula VI is: 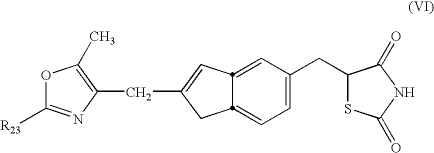
wherein R₂₃ is alkyl of 1 to 6 carbon atoms, cycloalkyl of 3 to 7 carbon atoms, phenyl or mono- or all-substituted phenyl wherein the substituents are independently alkyl of 1 to 6 carbon atoms, alkoxy of 1 to 3 carbon atoms, halogen, or trifluoromethyl.

12. The method of claim 1, the PPARγ agonist or a derivative thereof comprising a compound of Formula VII or pharmaceutically acceptable salt of a compound of Formula VII, wherein Formula VII is: 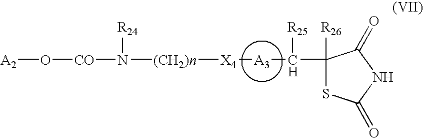
wherein A² represents an alkyl group, a substituted or unsubstituted aryl group, or an aralkyl group wherein the alkylene or the aryl moiety is substituted or unsubstituted;
A³ represents a benzene ring having in total up to 3 optional substituents;
R₂₄ represents a hydrogen atom, an alkyl group, an acyl group, an aralkyl group wherein the alkyl or the aryl moiety is substituted or unsubstituted, or a substituted or unsubstituted aryl group; or A² together with R₂₄ represents substituted or unsubstituted C₂₋₃ polymethylene group;
R₂₅ and R₂₆ each represent hydrogen, or R₂₅ and R₂₆ together represent a bond; X₄ represents O or S; and
n represents an integer in the range from 2 to 6.

13. The method of claim 1, the PPARγ agonist or a derivative thereof comprising a compound of Formula VIII or pharmaceutically acceptable salt of a compound of Formula VIII, wherein Formula VIII is: 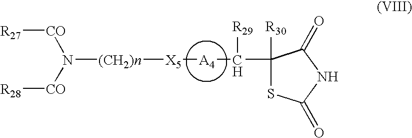
wherein: R₂₇ and R₂₈ each independently represent an alkyl group, a substituted or unsubstituted aryl group, or an aralkyl group being substituted or unsubstituted in the aryl or alkyl moiety;
or R₂₇ together with R₂₈ represents a linking group, the linking group consisting or an optionally substituted methylene group or an O or S atom; R₂₉ and R₃₀ each represent hydrogen, or R₂₉ and R₃₀ together represent a bond;
A₄ represents a benzene ring having in total up to 3 optional substituents;
X₅ represents O or S; and
n represents an integer in the range of 2 to 6.

14. The method of claim 1, the PPARγ agonist or a derivative thereof comprising a compound of Formula IX or pharmaceutically acceptable salt of a compound of Formula IX, wherein Formula IX is: 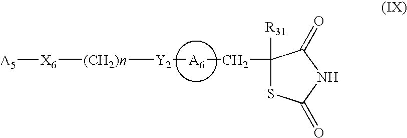
wherein: A₅ represents a substituted or unsubstituted aromatic heterocyclyl group; A₆ represents a benzene ring having in total up to 5 substituents;
X₆ represents O, S, or NR₃₂ wherein R₃₂ represents a hydrogen atom, an alkyl group, an acyl group, an aralkyl group, wherein the aryl moiety may be substituted or unsubstituted, or a substituted or unsubstituted aryl group;
Y₂ represents O or S;
R₃₁ represents an alkyl, aralkyl, or aryl group; and n represents an integer in the range from 2 to 6.

15. The method of claim 1, the PPARγ agonist or a derivative thereof comprising a compound of Formula X or pharmaceutically acceptable salt of a compound of Formula X, wherein Formula X is: 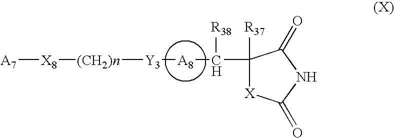
wherein: A₇ represents a substituted or unsubstituted aryl group;
A₈ represents a benzene ring having in total up to 5 substituents;
X₈ represents O, S, or NR₉, wherein R₃₉ represents a hydrogen atom, an alkyl group, an acyl group, an aralkyl group, wherein the aryl moiety may be substituted or unsubstituted, or a substituted or unsubstituted aryl group;
Y₃ represents O or S;
R₃₇ represents hydrogen;
R₃₈ represents hydrogen or an alkyl, aralkyl, or aryl group or R₃₇ together with R₃₈ represents a bond; and
n represents an integer in the range from 2 to 6.

16. The method of claim 1, the PPARγ agonist or a derivative thereof comprising a compound of Formula II or pharmaceutically acceptable salt of a compound of Formula XI, wherein Formula XI is: 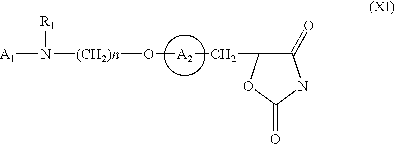
wherein A₁ represents a substituted or unsubstituted aromatic heterocyclyl group;
R¹ represents a hydrogen atom, an alkyl group, an acyl group, an aralkyl group, wherein the aryl moiety may be substituted or unsubstituted, or a substituted or unsubstituted aryl group;
A² represents a benzene ring having in total 1 up to 5 substituents; and n represents an integer in the range of from to 6.

17. The method of claim 1, the PPARγ agonist or a derivative thereof comprising a compound of Formula XII or Formula XIII or pharmaceutically acceptable salt of a compound of Formula XII or Formula XIII, wherein Formula XII and Formula XIII are: 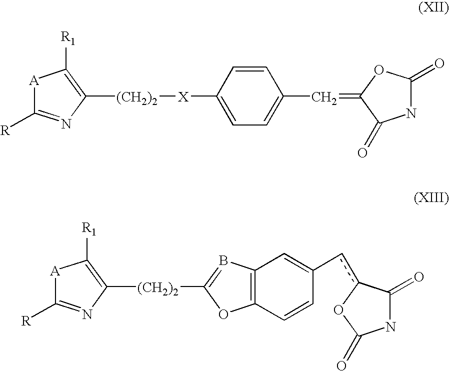
wherein the dotted line represents a bond or no bond;
R is cycloalkyl of three to seven carbon atoms, naphthyl, thienyl, furyl, phenyl, or substituted phenyl wherein the substituent is alkyl of one to three carbon atoms, alkoxy of one to three carbon atoms, trifluoromethyl, chloro, fluoro, or bis(trifluoromethyl);
R₁ is alkyl of one to three carbon atoms;
X is O or C═O;
A is O or S; and
B is N or CH.

18. The method of claim 1, the PPARγ agonist or a derivative thereof comprising at least one compound or a pharmaceutically salt thereof selected from the group consisting of:
(+)-5 [[4-[(3,4-dihydro-6-hydroxy-2,5,7,8-tetramethyl-2H-1-benzopyran-2-yl)methoxy]phenyl]methyl]-2,4thiazolidinedione; 5-[4-[2-(5-ethylpyridin-2-yl)ethoxyl]benzyl]thiazolidine-2,4-dione; 5-[4-[(1-methylcyclohexyl)methoxy]benzyl]thiazolidine-2,4-dione; (ciglitazone); 4-(2-naphthylmethyl)-1,2,3,5-oxathiadiazole-2-oxide; 5-[4-[2-[(N-(benzoxazol-2-yl)-N-methylamino]ethoxy]benzyl]-5-methlthiazolidine-2,4-dione; 5-[4-[2-[2,4diox-5-phenylthiazolidine-3-yl)ethoxy]benzyl]thiazolidine-2,4-dione; 5-[4-[2-[(N-methyl-N-(phenoxycarbonyl)amino]ethoxy]benzyl]thiazolidine-2,4-dione; 5-[4-[2-phenoxyethoxy)benzyl]thiazolidine-2,4-dione; 5-[4-[2-(4-chorophenyl)ethylsulfonyl]benzyl]thiazolidine-2,4-dione; 5-[4-[3-(5-methyl-2-phenyloxazol-4-yl)propionyl]benzyl]thiazolidine-2,4-dione; 5-[[4-(3-hydroxy-1-methylcyclohexyl)methoxy]benzyl]thiazolidine-2,4-dione; 5-[4-[2-(5-methyl-2-phenyloxazol-4-yl)ethoxyl]benzyl]thiazolidine-2,4-dione; 5-[(2-benzyl-2,3-dihydrobenzopyran)-5-ylmethyl]thiazolidine-2,4-dione; 5-[[2-(2-naphthylmethyl)benzoxazol]-5-ylmethyl]thiazolidine-2,4-dione; 5-[4-[2-(3-phenylureido)ethoxyl]benzyl]thiazolidine-2,4-dione; 5-[4-[2-(N-benzoxazol-2-yl)-N-metholamino]ethoxy]benzyl]thiazolidine-2,4-dione; 5-[4-[3-(5-methyl-2-phenyloxazol-4-yl)propionyl]benzyl]thiazolidine-2,4-dione; 5-[2-(5-methyl-2-phenyloxazol-4-ylmethyl)benzofuran-5-ylmethyl]oxazolidine-2,4-dione; 5-[4-[2-(N-methyl-N-(2-pyridyl)amino]ethoxy]benzyl]thiazolidine-2,4-dione; and 5-[4-[2-(N-(benzoxazol-2-yl)-N-methylamino]ethoxy]benzyl]oxazolidine-2,4-dione.

19. A method of treating glioma in a subject, the method comprising directly administering to the glioma a therapeutically effective amount of at least one PPARγ agonist or a derivative.

20. The method of claim 19, the PPARγ agonist or derivative thereof being administered to the subject in an amount effective to induce upregulation of proapoptotic protein Bax and cleaved caspase-3 associated.

21. The method of claim 19, the PPARγ agonist or derivative thereof being administered at an amount effective to suppress expression of MMP-9 in the brain of the subject.

22. The method of claim 19, the PPARγ agonist or derivative thereof being administered at an amount effective to mitigate glioma proliferation.

23. The method of claim 1, the PPARγ agonist or derivative thereof being administered directly to glioma of the subject by intracerebral infusion.

24. The method of claim 19, the PPARγ agonist or a derivative thereof comprising at least one compound or a pharmaceutically salt thereof selected from the group consisting of: (+)-5 [[4-[(3,4-dihydro-6-hydroxy-2,5,7,8-tetramethyl-2H-1-benzopyran-2-yl)ₘ ethoxy]phenyl]methyl]-2,4thiazolidinedione; 5-[4-[2-(5-ethylpyridin-2-yl)ethoxyl]benzyl]thiazolidine-2,4-dione; 5-[4-[(1-methylcyclohexyl)methoxy]benzyl]thiazolidine-2,4-dione; (ciglitazone); 4-(2-naphthylmethyl)-1,2,3,5-oxathiadiazole-2-oxide; 5-[4-[2-[(N-(benzoxazol-2-yl)-N-methylamino]ethoxy]benzyl]-5-methlthiazolidine-2,4-dione; 5-[4-[2-[2,4dioxo-5-phenylthiazolidine-3-yl)ethoxy]benzyl]thiazolidine-2,4-dione; 5-[4-[2-[(N-methyl-N-(phenoxycarbonyl)amino]ethoxy]benzyl]thiazolidine-2,4-dione; 5-[4-[2-phenoxyethoxy)benzyl]thiazolidine-2,4-dione; 5-[4-[2-(4-chorophenyl)ethylsulfonyl]benzyl]thiazolidine-2,4-dione; 5-[4-[3-(5-methyl-2-phenyloxazol-4-yl)propionyl]benzyl]thiazolidine-2,4-dione; 5-[[4-(3-hydroxy-1-methylcyclohexyl)methoxy]benzyl]thiazolidine-2,4-dione; 5-[4-[2-(5-methyl-2-phenyloxazol-4-yl)ethoxyl]benzyl]thiazolidine-2,4-dione; 5-[(2-benzyl-2,3-dihydrobenzopyran)-5-ylmethyl]thiazolidine-2,4-dione; 5-[[2-(2-naphthylmethyl)benzoxazol]-5-ylmethyl]thiazolidine-2,4-dione; 5-[4-[2-(3-phenylureido)ethoxyl]benzyl]thiazolidine-2,4-dione; 5-[4-[2-(N-benzoxazol-2-yl)-N-metholamino]ethoxy]benzyl]thiazolidine-2,4-dione; 5-[4-[3-(5-methyl-2-phenyloxazol-4-yl)propionyl]benzyl]thiazolidine-2,4-dione; 5-[2-(5-methyl-2-phenyloxazol-4-ylmethyl)benzofuran-5-ylmethyl]oxazolidine-2,4-dione; 5-[4-[2-(N-methyl-N-(2-pyridyl)amino]ethoxy]benzyl]thiazolidine-2,4-dione; and 5-[4-[2-(N-(benzoxazol-2-yl)-N-methylamino]ethoxy]benzyl]oxazolidine-2,4-dione.

25. A method of treating glioma in a subject, the method comprising directly administering to the glioma a therapeutically effective amount of at least one PPARγ agonist or a derivative, the PPARγ agonist or derivative thereof being administered to the subject in an amount effective to induce upregulation of proapoptotic protein Bax and cleaved caspase-3 associated, the PPARγ agonist or derivative thereof being administered directly to glioma of the subject by intracerebral infusion.
